(19) United States
(12) Patent Application Publication
(10) Pub. No.: US 2021/0267948 A1
(43) Pub. Date: Sep. 2, 2021

(54) TREATMENT OF CANCER

(71) Applicant: CITY OF HOPE, Duarte, CA (US)

(72) Inventors: Joseph Chao, Duarte, CA (US); David Horne, Duarte, CA (US); Paul Frankel, Duarte, CA (US)

(21) Appl. No.: 17/096,576

(22) Filed: Nov. 12, 2020

Related U.S. Application Data
(63) Continuation of application No. 16/483,379, filed on Aug. 2, 2019, now abandoned.
(60) Provisional application No. 62/455,430, filed on Feb. 6, 2017.
(60) Provisional application No. 62/511,747, filed on May 26, 2017.

(51) Int. Cl. A61K 31/426; A61P 35/02

(52) U.S. Cl. CPC A61K 31/426; A61P 35/02

(57) ABSTRACT

Provided herein, inter alia, are methods of treating cancer in a subject, by administering COH29 ((N-(4-(3,4-dihydroxyphenyl)-5-phenylthiazol-2-yl)-3,4-dihydroxybenzamide)).

### CROSS-REFERENCES TO RELATED APPLICATIONS

This application is a Continuation of U.S. patent application Ser. No. 16/483,379 filed Aug. 2, 2019, which is a 3.71 National Stage Entry of Application No. PCT/US18/17119 filed Feb. 6, 2018, which claims priority to U.S. Provisional Application No. 62/455,430 filed Feb. 6, 2017 and to U.S. Provisional Application No. 62/511,747 filed May 26, 2017, which are incorporated herein by reference in their entireties.

The sequence listing written in file 048440-648C01US Sequence Listing ST25, created on Nov. 12, 2020, 1,184 bytes, machine format IBM-PC, MS-Windows operating system, is hereby incorporated by reference in its entirety for all purposes.

### BACKGROUND

The antimetabolite drug hydroxyurea (HU) has been used to treat a variety of human cancers including chronic myelogenous leukemia, head and neck cancer, and others (1). Its primary anticancer target is ribonucleotide reductase (RR), which reduces ribonucleotides to their corresponding deoxy forms to supply dNTPs for DNA replication and repair (3,4). The human RR is composed of the hRRM1 and hRRM2 subunits (3,4). Following a genotoxic stimulus, an alternate RR enzyme is induced to supply dNTPs for DNA repair, which is composed of hRRM1 and p53R2 (a homologue of hRRM2 transactivated by the tumor suppressor protein p53) (5). Within cells, HU is known to inhibit both types of RR(4) through generating free radicals via oxidative transformation(6) that quenches free-radical mediated catalysis(3). However, pharmacologically, HU therapy suffers from short half-life in vivo and problematic side effects, most notably myelosuppression, and gastrointestinal and dermatologic effects (7).

Poly(ADP-ribose) polymerase-1 (PARP1) and PARP2 are both ADP-ribosyl transferases (ART) with roles in tumor development. ART members with PARP activity such as PARP1 contain a conserved catalytic domain with a highly conserved active site sequence (12-14). Following single strand DNA breaks PARP1 synthesizes ADP-ribose polymers from (3-NAD+ substrate and transfers these to glutamate, lysine or aspartate residues of acceptor proteins (itself or other proteins), which are subsequently degraded by poly(ADP-ribose) glycohydrolase (PARG). During single strand DNA break repair (SSBR) or base-excision repair (BER), PARP1 and PARP2 interact with X-ray repair complementing protein-1 (XRCC1) to recruit SSBR/BER factors, DNA polymerase β or DNA ligase III to the site of DNA damage (12-14). Without PARP1, the continuing presence of single strand breaks during DNA replication will lead to stalled replication forks, whose resolution require BRCA1 or BRCA2-mediated homologous repair (HR) (15,16). BRCA1 along with BRCA2 are tumor suppressor genes linked to the onset of familial breast cancers (11). In the absence of BRCA1, double strand breaks consequently accumulate, resulting in cell death via apoptosis. BRCA1/2-defective tumors may be sensitive to PARP1 inhibitors but may suffer from acquired resistance to PARP1 inhibitors. Thus, there is a need in the art for BRCA1/2-defective tumor treatments that avoid side effects and/or acquired resistance associated with current therapies. Accordingly, provided herein are solutions to these and other problems in the art.

### BRIEF SUMMARY

In one aspect, there is provided a method of treating cancer in a subject in need thereof. The method includes administering an effective amount of a compound having the structure:

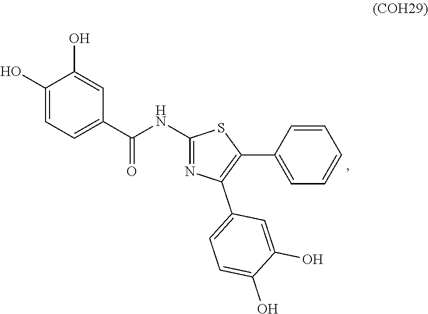 [SMILES: O=C(Nc1nc(-c2ccc(O)c(O)c2)c(-c2ccccc2)s1)c1ccc(O)c(O)c1]

wherein the effective amount is at least about 50 mg per day of administration.

In another aspect, there is provided a pharmaceutical composition including a pharmaceutically acceptable excipient and a compound having the structure:

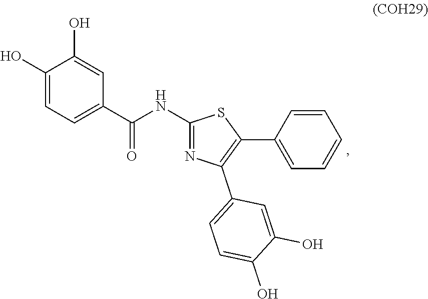 [SMILES: O=C(Nc1nc(-c2ccc(O)c(O)c2)c(-c2ccccc2)s1)c1ccc(O)c(O)c1]

wherein the compound is present in an amount of from about 50 mg to about 1000 mg.

In another aspect, there is provided a kit comprising a dispensing apparatus configured to dispense the pharmaceutical composition disclosed herein each day for 21 days followed by 7 days of no administration of the pharmaceutical composition.

### BRIEF DESCRIPTION OF THE DRAWINGS

FIGS. 1A-1C. BRCA1 status affects COH29 cytotoxicity and antitumor activity: FIG. 1A: Dose response curves for ovarian cancer cells expressing wt (wild-type) BRCA1 (0V90) or mutant BRCA1 (UWB1.289) incubated with COH29 for 72 h, and lysed (cell viability was assessed by MTT assay), and the points depicted represent an average of three independent experiments with error bars indicated; growth of tumor explants established with HCC1937 (FIG. 1B) and HCC1937+BRCA1 (FIG. 1C) cells in the mammary fat pads of female NSG mice (mice were treated with COH29 or vehicle as indicated, results are the mean±standard error of tumor measurements from 4 mice/group).

FIGS. 2A-2B. Correlation of RRM2 expression with PARP1 in patient cohorts. Regression plots of the expression of RRM2 and PARP1 extracted from public databases of clinical outcomes in breast cancer (FIG. 2A) and ovarian cancer (FIG. 2B).

FIGS. 3A-3B. COH29 inhibits PARP1 in BRCA1-defective human breast cancer cells. (FIG. 3A): the effect of COH29 on PARP1 activity in isogenic pairs of breast (HCC1937) and ovarian (UWB1.289) cancer cell lines expressing mutant or wt BRCA1 (wt=+BRCA1 in each case) was assessed in duplicate using procedures described in Materials & Methods; FIG. 3B: the effect of COH29 on PARP1 protein expression in the isogenic HCC1937/HCC1937+BRCA1 cell lines was assessed by Western blot analysis using anti-human PARP1 antibody as the primary antibody. Loading control is β-actin.

FIGS. 4A-4B. The effect of BRCA1 on cell survivability following the dual treatment with COH29 and cisplatin. FIG. 4A: viability of HCC1937 and HCC1937+BRCA1 cells treated with a fixed concentration of COH29 (12.5 μM) plus cisplatin (12.5, 25, 50 and 100 μM) for 24 h assessed by MTT assay (the points depicted represent an average of three independent experiments with error bars indicated); FIG. 4B: histogram of 24 h viability in the cells indicated in the presence of 5 μM COH29 alone, 4 μM cisplatin alone, or the combination of the two drugs at the same concentrations (shown are the averages of three independent experiments).

FIGS. 5A-5D. Effect of COH29 compared to HU in zebrafish genotoxicity assay. FIG. 5A: wild-type zebrafish embryos at 4 dpf (day post-fertilization) exposed to HU as indicated (morphological changes in the eye and heart development are indicated by the arrowheads).

FIG. 5B: bar graph of the effect of a series of different concentrations of HU on zebrafish (0, 5, 10, 20, 50 mM, n=50, performed in triplicate). FIG. 5C: wild-type zebrafish embryos at 4 dpf exposed to COH29 as indicated. FIG. 5D: bar graph of the effect of a series of different concentrations of COH29 on zebrafish (0, 10, 20, 50, 100 μM, n=46, performed in triplicate).

FIG. 6. COH29 treatment activates DNA damage checkpoint. The effect of COH29 treatment on DNA damage checkpoint proteins in human breast cancer cells expressing wt p53 (MCF-7) or defective for p53 (MCF-7p53−/−), and in triple-negative breast cancer cells (MDA-MB-468) assessed by Western Blot.

FIGS. 7A-7D. COH29 activates DDR and suppresses RAD51 expression in BRCA1 wild-type human lung cancer cells. FIG. 7A: the effect of COH29 on DDR-associated proteins were assessed in cytoplasm and nucleus by Western blot analysis, where cells were treated with COH29 at the indicated doses for 48 h and cell lysates were subjected to immunoblotting using the indicated antibodies (FOXO3 activity is indicated by the levels of its downstream target p27Kip1 and β-Tubulin and Lamin A/C represents the fractionation and loading controls of Cyt. and Nuc. Extracts); FIGS. 7B-7D: the effect of COH29 on colocalization of DDR-related proteins, phospho-ATM (FIG. 7B), γ-H2AX (FIG. 7C), and phospho-p53 (FIG. 7D) and foxo3 in the nucleus was assessed by indirect immunofluorescence assay. For each protein, an average of 300 of the stained cells was analyzed and a histogram shows the percentage (%) of cells with positive nuclei (≥5 foci) where the number of biological replicates is three, the error bars represent standard deviation (SD) and P values (paired t-test) are as indicated).

FIGS. 8A-8B. COH29 effect on NHEJ DNA repair. The activity of COH29 alone, or in combination with cisplatin at the doses shown, assessed by FACS analysis of EJ2 (FIG. 8A) (alternative NHEJ pathway) or EJ5 (FIG. 8B) (NHEJ pathway) cells after 24 h exposure of the cells to the drugs.

FIGS. 9A-9B. COH29 suppresses RAD51 in human lung cancer cells. FIG. 9A: the effect of COH29 on RAD51 protein was assessed by indirect immunofluorescence assay using anti-human RAD51 antibody as the primary antibody. FIG. 9B: the effect of COH29 on RAD51 protein was assessed by Western blot analysis using anti-human RAD51 antibody as the primary antibody (loading control was (3-actin), for the analysis; A549 lung cancer cells were treated with COH29 at the indicated doses for 24 h and the expression pattern of γ-H2AX following the COH-29 treatment was also similarly analyzed in FIGS. 9A and 9B.

FIG. 10 depicts a capability of COH29 to bind a pocket on the surface of the M2 subunit of ribonucleotide reductase (RR).

FIGS. 11A-11B depict the activity of COH29 in gemcitabine (KB-Gem) (FIG. 11A) and hydroxyurea (KBHURs) (FIG. 11B) resistant cell lines in 72 h toxicity assay. Hydroxyurea (concentration 2-50 mmol/L), gemcitabine (concentration 20-500 μmon), and COH29 (concentration 2-250 μmon) were used for real-time growth curves for KBHUR and gemcitabine, respectively, based on comparison of 72-h viability of hydroxyurea-resistant or gemcitabine-resistant cells to COH29.

FIGS. 12A-12D depict the activity of COH29 in mouse xenografts. Cancer cells were implanted and allowed to grow at the subcutaneous site until the tumor was measurable. Subcutaneous xenograft growth curves for leukemic-(MOLT-4) mice treated for 12 days with oral COH29 in twice-daily dosing at 50 and 100 mg/kg (FIG. 12A) and for ovarian cancer bearing mice (TOV112D) treated for 7 days with 200, 300, or 400 mg/kg of oral COH29 (FIG. 12B) (shown are the average±SDs for 4 animals/group). FIG. 12C depicts the activity of RNR in MOLT-4 tumor xenografts from mice treated with vehicle (solutol-15) or 100 mg/kg of oral COH29 for 12 days. FIG. 12D depicts the effect of oral COH29 on intratumoral dTNP pools from MOLT-4 tumor xenografts.

FIGS. 13A-13B depict the effect of COH29 in HCC1937 BRCA1-deficient and HCC1937 BRCA1 wild type breast cancer cells in vitro (FIG. 13A) and in mouse mammary fat pad othotopic tumor xenografts (FIG. 13B).

FIG. 14 depicts the experimental design scheme of the clinical protocol of COH29. COH29 is administered orally once or twice daily, depending on dose levels for 21 days. Time course of PK sampling begins prior to the first dose of cycle 1, and then at 15 min, 30 min, 1 h, 2 h, 3 h, 4 h, 6 h, 8 h, 24 h (i.e. prior to the morning dose on day 2), and 168 hours (i.e. prior to the morning dose on day 8) following the first COH29 dose. Blood sampling occurs prior to study drug administration and pre dosing on day 1 of subsequent cycles, where applicable, and is performed for PBMC PD studies at the end of the study.

FIG. 15 depicts the accelerated titration phase I design for COH29 (based on dose limiting toxicity (“DLT”)).

### DETAILED DESCRIPTION

### Definitions

“Patient,” “subject,” “patient in need thereof,” and “subject in need thereof” are herein used interchangeably and refer to a living organism suffering from or prone to a disease or condition that may be treated by administration of a COH29 or COH29 in combination with other anti-cancer agents as discussed herein. In embodiments, the disease or condition is cancer. Non-limiting examples of subjects include humans, other mammals, bovines, rats, mice, dogs, monkeys, goat, sheep, cows, deer, and other non-mammalian animals. In embodiments, a patient is human.

A “cancer subject” as used herein refers to a subject who has a cancer as described herein. A cancer subject may have at least one of the cancers described herein. Thus, for example, a cancer subject may refer to a “breast cancer subject” (e.g. a subject having breast cancer) or an “ovarian cancer subject” (e.g. a subject having ovarian cancer). Cancer subjects may have cancers that exhibit specific genotypic or phenotypic characteristics (e.g. defective gene products or resistance to specific anti-cancer agents). Accordingly, a cancer subject may be a “BRCA1-defective subject” where a BRCA1-defective subject is a subject who has a cancer that includes a BRCA1 defective gene or BRCA1 defective protein (e.g. a “BRCA1-defect”). In embodiments, a “BRCA1-defective subject” refers to the non-expression (e.g. reduced expression relative to control or healthy subjects) of the BRCA1 gene, absence of (e.g. reduced amount relative to control or healthy subjects) functional BRCA1 in the subject or reduced expression of a BRCA1 that causes, at least in part, directly or indirectly, cancer in the subject. In embodiments, a BRCA1-defective subject displays non-expression of the BRCA1 gene, absence of functional BRCA1 in the subject. A cancer subject may be a “PARP1 inhibitor-resistant subject” where a PARP1 inhibitor-resistant subject is a subject who has a cancer resistant to at least one PARP1 inhibitor as known in the art. A cancer subject may be a “DNA-damaging anti-cancer agent resistant subject” where such a subject has a cancer resistant to at least one DNA-damaging anti-cancer agent as known in the art. Cancer subjects may have cancers that exhibit more than one genotypic or phenotypic characteristic (e.g. a breast cancer subject may have a cancer that has a BRCA1-defect and resistance to at least one PARP1 inhibitor).

“COH29” refers to a compound having formula (N-(4-(3,4-dihydroxyphenyl)-5-phenylthiazol-2-yl)-3,4-dihydroxybenzamide):

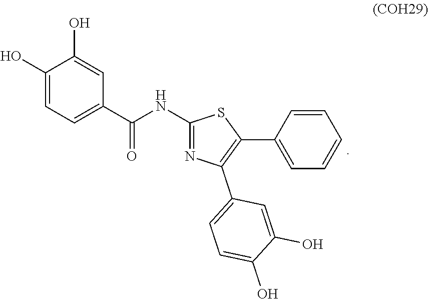 [SMILES: O=C(Nc1nc(-c2ccc(O)c(O)c2)c(-c2ccccc2)s1)c1ccc(O)c(O)c1]

COH29 and its synthesis are described in U.S. Pat. Nos. 7,956,076; 8,372,983, and International Application No.: PCT/US13/24490 which are herein incorporated by reference in their entireties.

COH29 may be administered to cancer subjects described herein, including for example, a breast cancer subject, an ovarian cancer subject, a BRCA1-defective subject, a PARP1 inhibitor-resistant subject or a DNA-damaging anti-cancer agent resistant subject. The administration may be at a therapeutically effective amount as set forth herein.

“BRCA1” is used according to its common, ordinary meaning and refers to proteins of the same or similar names and functional fragments and homologs thereof. The term includes recombinant or naturally occurring forms of BRCA1 (e.g. breast cancer 1, early onset; GI No: 1698399), or variants thereof that maintain BRCA1 activity (e.g. within at least 30%, 40%, 50%, 60%, 70%, 80%, 90%, 95%, or 100% activity compared to BRCA1).

“γ-H2AX” is used according to its common, ordinary meaning and refers to proteins of the same or similar names and functional fragments and homologs thereof. The term includes any recombinant or naturally occurring form of γ-H2AX (e.g. γ histone H2AX; GI No: 4504253), or variants thereof that maintain γ-H2AX activity (e.g. within at least 30%, 40%, 50%, 60%, 70%, 80%, 90%, 95%, or 100% activity compared to γ-H2AX).

“Rad51” is used according to its common, ordinary meaning and refers to proteins of the same or similar names and functional fragments and homologs thereof. The term includes any recombinant or naturally occurring form of Rad51 (e.g. GI No: 49168602), or variants thereof that maintain Rad51 activity (e.g. within at least 30%, 40%, 50%, 60%, 70%, 80%, 90%, 95%, or 100% activity compared to Rad51).

“PARP1” is used according to its common, ordinary meaning and refers to proteins of the same or similar names and functional fragments and homologs thereof. The term includes any recombinant or naturally occurring form of PARP1 (e.g. poly [ADP-ribose] polymerase 1; GI No: 156523968)), or variants thereof that maintain PARP1 activity (e.g. within at least 30%, 40%, 50%, 60%, 70%, 80%, 90%, 95%, or 100% activity compared to PARP1). A “PARP1 inhibitor” is a composition (e.g. compound, peptide, protein, nucleic acid, or antibody) which inhibits the activity of PARP1 (NAD⁺ ADP-ribosyltransferase 1).

PARP1 inhibitors” are compositions (e.g. a compound, polypeptide, amino acid, polynucleotide, nucleic acid, or antibody) effective at treating cancers by inhibiting the activity of, or the expression of PARP1. Non-limiting examples of PARP1 inhibitors include olaparib, veliparib, iniparib, and niraparib.

“DNA-damaging anti-cancer agents” are compositions (e.g. a compound, polypeptide, amino acid, polynucleotide, nucleic acid, or antibody) effective at treating cancers by damaging DNA. DNA-damaging anti-cancer agents may be chemotherapeutic. In embodiments, DNA-damaging agents include irradiation (e.g. γ-irradiation). The interaction of a DNA-damaging anti-cancer agent may be direct (e.g. binding or interacting with DNA itself) or indirect (e.g. binding or interacting with other molecules interacting with DNA). Herein, DNA-damaging anti-cancer agents include, for example, alkylating agents (e.g. ethylenimines, methylmelamines, nitrosoureas, nitrogen mustards, busulfans, cyclophosphamides, and procarbazines), antimetabolites, anthracyclines, platinum based agents, taxanes, kinase inhibitors, histone deacetylase inhibitors (HDAC), topoisomerase inhibitors, and nucleotide analogues. In embodiments, DNA-damaging anti-cancer agents include compositions that intercalate between DNA base pairs or bind in the minor or major grooves of a DNA. In embodiments, the DNA-damaging anti-cancer agents is a Topoisomerase I agent, camptothecin, irinotecan, topotecan, a Topoisomerase II agent, cisplatin, carboplatin, oxaliplatin, adriamycin (e.g., doxorubicin), etoposide, a single-strand break agent (e.g. BCNU (carmustine), CCNU (lomustine)), DTIC (dacarbazine), cytoxan (cyclophosphamide), ifosfamide, bleomycin, and mitomycin C.

“Chemotherapeutic” or “chemotherapeutic agent” is used in accordance with its plain ordinary meaning and refers to a chemical composition or compound having antineoplastic properties or the ability to inhibit the growth or proliferation of cells.

The anticancer drug cisplatin has been used to treat various human cancers including, for example, ovarian cancer, testicular cancer, germ cell tumors, small cell lung cancer, lymphomas, head and neck cancer, and bladder cancer. Herein, a “platinum-based compound” or “platinum containing agent” as refers to a compound comprising a heavy metal complex containing a central atom of platinum surrounded by organic and/or inorganic functionalities. Included within platinum-based compounds are platinum-based drugs. Non-limiting examples of platinum-based compounds include, cisplatin, carboplatin, nedaplatin, oxaliplatin, satraplatin, triplatin, tetranitrate, pharmaceutically acceptable salts thereof, stereoisomers thereof, derivatives thereof, analogues thereof, and combinations thereof. The term “cisplatin” includes derivatives and analogues such as those described in U.S. Pat. Nos. 4,177,263, 4,584,316, 5,648,362 and 5,399,694, which are herein incorporated by reference in their entirety.

Cisplatin anticancer activity stems primarily from the crosslinking of DNA in target cells, which requires an exchange reaction involving cisplatin chloride ions with nucleophile groups. Cisplatin causes bidentate lesions in DNA through formation of intrastrand adducts with d(GpG) or d(ApG) sequences. Cisplatin is also capable of generating interstrand crosslinks, which may interfere with DNA replication. The lesions activate the DNA damage checkpoint, resulting in the arrest of cell cycle progression. The formation of secondary tumors in patients represents one of the major issues associated with cisplatin therapy. Other side effects of cisplatin may include nephrotoxicity, neurotoxicity, nausea, ototoxicity, myelotoxicity, and electrolyte imbalance. Cisplatin resistance is also found in cancer patients.

The terms “treating” or “treatment” refers to any indicia of success in the treatment or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms may be based on objective or subjective parameters; including the results of a physical examination, neuropsychiatric exams, and/or a psychiatric evaluation. The term “treating” and conjugations thereof, include prevention of an injury, pathology, condition, or disease.

As used herein, the term “cancer” refers to all types of cancer, neoplasm, malignant or benign tumors found in mammals, including leukemia, carcinomas and sarcomas. Exemplary cancers include breast cancer, ovarian cancer, colon cancer, liver cancer, kidney cancer and pancreatic cancer. Additional examples include leukemia (e.g. acute myeloid leukemia (“AML”) or chronic myelogenous leukemia (“CML”)), cancer of the brain, lung cancer, non-small cell lung cancer, melanoma, sarcomas, and prostate cancer, cervix cancers, stomach cancers, head & neck cancers, uterus cancers, mesothelioma, metastatic bone cancer, medulloblastoma, Hodgkin's Disease, Non-Hodgkin's Lymphoma, multiple myeloma, neuroblastoma, rhabdomyosarcoma, primary thrombocytosis, primary macroglobulinemia, primary brain tumors, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, endometrial cancer, adrenal cortical cancer, neoplasms of the endocrine and exocrine pancreas.

The term “leukemia” refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). The murine leukemia model is widely accepted as being predictive of in vivo anti-leukemic activity. It is believed that a compound that tests positive in the P388 cell assay will generally exhibit some level of anti-leukemic activity regardless of the type of leukemia being treated. Accordingly, the present disclosure includes a method of treating leukemia, including treating acute myeloid leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, and undifferentiated cell leukemia.

The term “sarcoma” generally refers to a tumor which is made up of a substance like the embryonic connective tissue and is generally composed of closely packed cells embedded in a fibrillar or homogeneous substance. Sarcomas which may be treated with a combination of antineoplastic thiol-binding mitochondrial oxidant and an anticancer agent include a chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, and telangiectaltic sarcoma.

The term “melanoma” is taken to mean a tumor arising from the melanocytic system of the skin and other organs. Melanomas which may be treated with a combination of antineoplastic thiol-binding mitochondrial oxidant and an anticancer agent include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, and superficial spreading melanoma.

The term “carcinoma” refers to a malignant new growth made up of epithelial cells tending to infiltrate the surrounding tissues and give rise to metastases. Exemplary carcinomas which may be treated with a combination of antineoplastic thiol-binding mitochondrial oxidant and an anticancer agent include, for example, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, Comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniforni carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypemephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, and carcinoma villosum.

“Cancer model organisms” are organisms (e.g. cancer cell lines) exhibiting a phenotype indicative of cancer or the activity of cancer causing elements, within the organism. The cancer model organism may exhibit a phenotype of a cancer as described herein. Accordingly, a cancer model organism may be, for example, a cancer cell line deficient in BRCA1 that is resistant to a PARP1 inhibitor, or is resistant to a DNA-damaging anti-cancer agent. A wide variety of organisms may serve as cancer model organisms, and include for example, cancer cells and mammalian organisms such as rodents (e.g. mouse or rat) and primates (such as humans). Cancer cell lines are widely understood by those skilled in the art as cells exhibiting phenotypes or genotypes similar to in vivo cancers. Cancer cell lines as used herein include cell lines from animals (e.g. mice) and from humans.

An “anti-cancer agent” used in accordance with its plain ordinary meaning and refers to a composition (e.g. a compound, polypeptide, amino acid, polynucleotide, nucleic acid, or antibody) having antineoplastic properties or the ability to inhibit the growth or proliferation of cells. In some embodiments, an anti-cancer agent is a chemotherapeutic. In some embodiments, an anti-cancer agent is an agent identified herein having utility in methods of treating cancer. In some embodiments, an anti-cancer agent is an agent approved by the FDA or similar regulatory agency of a country other than the USA, for treating cancer. Anti-cancer agents may be selective for certain cancers or certain tissues.

As used herein, the term “administering” means oral administration, administration as a suppository, topical contact, intravenous, parenteral, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc.

By “co-administer” it is meant that a composition described herein is administered at the same time, just prior to, or just after the administration of one or more additional therapies. For example, COH29 may be administered alone or may be co-administered to the patient. Co-administration is meant to include simultaneous or sequential administration of the compound individually or in combination (more than one compound or agent). Thus, the preparations may also be combined, when desired, with other active substances (e.g. to reduce metabolic degradation).

The compositions disclosed herein can be delivered transdermally, by a topical route, formulated as applicator sticks, solutions, suspensions, emulsions, gels, creams, ointments, pastes, jellies, paints, powders, and aerosols. Oral preparations include tablets, pills, powder, dragees, capsules, liquids, lozenges, cachets, gels, syrups, slurries, suspensions, etc., suitable for ingestion by the patient. Solid form preparations include powders, tablets, pills, capsules, cachets, suppositories, and dispersible granules. Liquid form preparations include solutions, suspensions, and emulsions, for example, water or water/propylene glycol solutions. The compositions of the present invention may additionally include components to provide sustained release and/or comfort. Such components include high molecular weight, anionic mucomimetic polymers, gelling polysaccharides and finely divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes. The compositions disclosed herein can also be delivered as microspheres for slow release in the body. For example, microspheres can be administered via intradermal injection of drug-containing microspheres, which slowly release subcutaneously (see Rao, J. Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). In another embodiment, the formulations of the compositions of the present invention can be delivered by the use of liposomes which fuse with the cellular membrane or are endocytosed, i.e., by employing receptor ligands attached to the liposome, that bind to surface membrane protein receptors of the cell resulting in endocytosis. By using liposomes, particularly where the liposome surface carries receptor ligands specific for target cells, or are otherwise preferentially directed to a specific organ, one can focus the delivery of the compositions of the present invention into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989). The compositions can also be delivered as nanoparticles.

In some embodiments, two or more different pharmaceutical compositions are co-administered. In some instances, the two or more different pharmaceutical compositions are co-administered simultaneously. In some cases, the two or more different pharmaceutical compositions are co-administered sequentially without a gap of time between administrations. In other cases, the two or more different pharmaceutical compositions are co-administered sequentially with a gap. In certain embodiments, the gap between co-administration of two or more different pharmaceuticals may be about 0.25 hour, about 0.5 hour, about 1 hour, about 2 hours, about 3 hours, about 12 hours, about 1 day, about 2 days, or more between administration of the different pharmaceuticals.

An “effective amount” is an amount sufficient for a compound to accomplish a stated purpose relative to the absence of the compound (e.g. achieve the effect for which it is administered, treat a disease, reduce enzyme activity, increase enzyme activity, reduce a signaling pathway, or reduce one or more symptoms of a disease or condition). An example of a “therapeutically effective amount” is an amount sufficient to contribute to the treatment, prevention, or reduction of a symptom or symptoms of a disease, which could also be referred to as a “therapeutically effective amount.” A “reduction of” a symptom or symptoms (and grammatical equivalents of this phrase) means decreasing of the severity or frequency of the symptom(s), or elimination of the symptom(s). The exact amounts may depend on the purpose of the treatment, and may be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

The dosage and frequency (single or multiple doses) administered to a mammal may vary depending upon a variety of factors, for example, whether the mammal suffers from another disease, and its route of administration; size, age, sex, health, body weight, body mass index, and diet of the recipient; nature and extent of symptoms of the disease being treated, kind of concurrent treatment, complications from the disease being treated or other health-related problems. Other therapeutic regimens or agents may be used in conjunction with the methods and compounds of this disclosure. Adjustment and manipulation of established dosages (e.g., frequency and duration) are well within the ability of those skilled in the art.

The therapeutically effective amounts described herein may be initially determined from cell culture assays. Target concentrations may be those concentrations of active compound(s) that are capable of achieving the methods described herein, as measured using the methods described herein or known in the art.

As is well known in the art, therapeutically effective amounts for use in humans may also be determined from animal models. For example, a dose for humans may be formulated to achieve a concentration that has been found to be effective in animals. The dosage in humans may be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan.

Dosages may be varied depending upon the requirements of the patient and the compound being employed. The dose administered to a patient, in the context of the present disclosure should be sufficient to affect a beneficial therapeutic response in the patient over time. The size of the dose also may be determined by the existence, nature, and extent of any adverse side effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. Dosage amounts and intervals may be adjusted individually to provide levels of the administered compound effective for the particular clinical indication being treated. This may provide a therapeutic regimen that is commensurate with the severity of the individual's disease state.

As defined herein, the terms “inhibition,” “inhibit,” “inhibiting” and the like in reference to a protein-inhibitor interaction means negatively affecting (e.g. decreasing) the activity or function of the protein relative to the activity or function of the protein in the absence of the inhibitor. When used in reference to a inhibiting a gene, “inhibiting” means negatively affecting (e.g. decreasing) the activity or expression of the gene relative to the activity or expression of the gene in the absence of the inhibitor. In some embodiments inhibition refers to reduction of a disease or symptoms of disease. In some embodiments, inhibition refers to a reduction in the activity of a particular protein or nucleic acid target. Thus, inhibition includes, at least in part, partially or totally blocking stimulation, decreasing, preventing, or delaying activation, or inactivating, desensitizing, or down-regulating signal transduction or enzymatic activity or the amount of a protein.

The terms “synergy,” “synergism,” “synergistic,” “combined synergistic amount” and “synergistic therapeutic effect” are used herein interchangeably and refer to a measured effect of compounds administered in combination where the measured effect is greater than the sum of the individual effects of each of the compounds administered alone.

The term “pharmaceutical composition” refers to a mixture of a compound COH29 that contains other chemical components. In some instances, additional chemical components are carriers, stabilizers, diluents, dispersing agents, suspending agents, thickening agents, and/or excipients. The pharmaceutical composition facilitates administration of the compound to an organism. Techniques of administering a compound include, but are not limited to, intravenous, oral, aerosol, parenteral, ophthalmic, pulmonary, and topical administration.

The term “adverse effect or side effect” refers to an undesirable secondary effect which occurs in addition to the desired therapeutic effect of a drug or medication. When severe, adverse effects may lead to non-compliance with prescribed treatment.

The term “dose limiting toxicity, or DLT” refers to a toxicity that is monitored while a patient is subjected to a treatment with a drug during the drug trial, and is part of a safety analysis. It refers to any of the following: grade 4 thrombocytopenia (platelet count<25,000/mm 3); grade 3 thrombocytopenia (platelet count 25,000-50,000/mm 3), associated with bleeding or need for transfusion; febrile neutropenia (per NCI CTC, absolute neutrophil count (ANC)<1.0×10 9/L and fever>38.5° C.); and any other>grade 3 non-hematologic toxicity considered by the investigator to be clinically significant and related to study drug.

The term “maximum tolerated dose, or MTD” refers to a dose that produces an “acceptable” level of toxicity or that, if exceeded, would put animals or patients at “unacceptable” risk for toxicity, and is the main objective of Phase I clinical trials in cancer and HIV treatment in which relatively high doses of drugs are chosen to achieve the greatest possible beneficial antitumor effect. The MTD can also refer to a dose that produces a certain frequency of DLT within the treated patient population.

The term “human equivalent dose or human equivalent concentration (HEC)” refers to quantity of a chemical (drug) that, when administered to humans, produces an effect equal to that produced in test animals by a smaller dose.

The term “objective tumor response” refers to a common endpoint in clinical trials to evaluate the efficacy of an anti-cancer agent, and encompasses several terms, such as objective response rate (ORR), time to progression (TTP), disease free survival (DFS), and progression free survival (PFS).

The term “biomarker” refers to a measurable indicator of the severity or presence of a disease state in an individual. Once a proposed biomarker has been validated, it can be used to diagnose disease risk, presence of disease in an individual, or to tailor treatments for the disease in an individual (choices of drug treatment or administration regimes). In evaluating potential drug therapies, a biomarker may be used as a surrogate for a natural endpoint such as survival or irreversible morbidity. If a treatment alters the biomarker, which has a direct connection to improved health, the biomarker serves as a surrogate endpoint for evaluating clinical benefit. The main area of use is in the drug development process.

### Methods

In a first aspect provided herein is a method of treating cancer in a subject in need thereof. The method includes administering an effective amount of COH29 to the subject. The subject is a BRCA1-defective subject, a PARP1 inhibitor-resistant subject or a DNA-damaging anti-cancer agent resistant subject as set forth herein. Thus, in embodiments, the subject is a BRCA1-defective subject. In embodiments, the subject may be a PARP1 inhibitor-resistant subject. In embodiments, the subject may be a DNA-damaging anti-cancer agent resistant subject. In embodiments, the subject is at least one of a BRCA1-defective subject, a PARP1 inhibitor-resistant subject or a DNA-damaging anti-cancer agent resistant subject. In embodiments, the subject may be a BRCA1-defective subject and at least one of PARP1 inhibitor-resistant subject or a DNA-damaging anti-cancer agent resistant subject (i.e. the cancer has a BRCA1-defect and resistance to at least one of a PARP1 inhibitor or a DNA-damaging anti-cancer agent). In embodiments, the cancer is a gemcitabine resistant cancer. In embodiments, the cancer is a hydroxyurea resistant cancer.

In embodiments, the subject is a breast cancer subject, ovarian cancer subject, colon cancer subject, liver cancer subject, kidney cancer subject, lung cancer subject, non-small cell lung cancer subject, brain cancer subject, prostate cancer subject, pancreatic cancer subject, melanoma subject, leukemia subject, or sarcoma subject.

In embodiments, the subject may be a breast cancer subject or an ovarian cancer subject. In embodiments, the subject may be a breast cancer subject. In embodiments, the subject may be an ovarian cancer subject. In embodiments, the subject may be a colon cancer subject. In embodiments, the subject may be a liver cancer subject. In embodiments, the subject may be a kidney cancer subject. In embodiments, the subject may be a lung cancer subject or a non-small cell lung cancer subject. In embodiments, the subject may be a brain cancer subject. In embodiments, the subject may be a prostate cancer subject. In embodiments, the subject may be a pancreatic cancer subject. In embodiments, the subject may be a melanoma subject. In embodiments, the subject may be a leukemia subject. In embodiments, the subject may be a sarcoma subject.

The cancer subject (e.g. breast, ovarian, lung, prostate, or pancreatic cancer subject) may also be least one of a BRCA1-defective subject, a PARP1 inhibitor-resistant subject, or a DNA-damaging anti-cancer agent resistant subject. Thus in embodiments, the cancer subject is a BRCA1-defective subject. In embodiments, the cancer subject is a PARP1 inhibitor-resistant subject. In embodiments, the cancer subject is a DNA-damaging anti-cancer agent resistant subject. In embodiments, the cancer subject is a BRCA1-defective subject and a PARP1 inhibitor-resistant subject. In embodiments, the cancer subject is a PARP1 inhibitor-resistant subject and a DNA-damaging anti-cancer agent resistant subject. In embodiments, the cancer subject is a BRCA1-defective subject, a PARP1 inhibitor-resistant subject and a DNA-damaging anti-cancer agent resistant subject.

Thus, in embodiments, the subject is a BRCA1-defective subject having breast cancer or ovarian cancer. The BRCA1-defective subject may have breast cancer. The BRCA1-defective subject may have ovarian cancer.

In embodiments, the subject is a PARP1 inhibitor-resistant subject having breast cancer or ovarian cancer. The PARP1 inhibitor-resistant subject may have breast cancer. The PARP1 inhibitor-resistant subject may have ovarian cancer.

In embodiments, the subject is a DNA-damaging anti-cancer agent resistant subject having a cancer characterized by resistance to at least one DNA-damaging anti-cancer agent including, but not limited to, cisplatin, carboplatin, oxaliplatin, adriamycin, mitoxantrone, VP16, CPT11, or camptothecin. In embodiments, the subject is a DNA-damaging anti-cancer agent resistant subject having breast cancer, ovarian cancer, colon cancer, liver cancer, kidney cancer, lung cancer, non-small cell lung cancer, brain cancer, prostate cancer, pancreatic cancer, melanoma, leukemia, or sarcoma. The subject may be a DNA-damaging anti-cancer agent resistant subject having breast cancer. The subject may be a DNA-damaging anti-cancer agent resistant subject having ovarian cancer,

In embodiments, the subject is a BRCA1-defective subject and a PARP1 inhibitor-resistant subject. The subject may be a BRCA1-defective subject and a PARP1 inhibitor-resistant subject having breast cancer or ovarian cancer. The subject may be a BRCA1-defective subject and a PARP1 inhibitor-resistant subject having breast cancer. The subject may be a BRCA1-defective subject and a PARP1 inhibitor-resistant subject having ovarian cancer.

In embodiments, the subject is a BRCA1-defective subject and a DNA-damaging anti-cancer agent resistant subject. The subject may be a BRCA1-defective subject and a DNA-damaging anti-cancer agent resistant subject having breast cancer or ovarian cancer. The subject may be a BRCA1-defective subject and a DNA-damaging anti-cancer agent resistant subject having breast cancer. The subject may be a BRCA1-defective subject and a DNA-damaging anti-cancer agent resistant subject having ovarian cancer.

In embodiments, the subject is a PARP1 inhibitor-resistant subject and a DNA-damaging anti-cancer agent resistant subject. The subject may be a PARP1 inhibitor-resistant subject and a DNA-damaging anti-cancer agent resistant subject having breast cancer or ovarian cancer. The subject may be a PARP1 inhibitor-resistant subject and a DNA-damaging anti-cancer agent resistant subject having breast cancer. The subject may be a PARP1 inhibitor-resistant subject and a DNA-damaging anti-cancer agent resistant subject having ovarian cancer.

In embodiments, the subject is a BRCA1-defective subject, a PARP1 inhibitor-resistant subject and a DNA-damaging anti-cancer agent resistant subject. The subject may be a BRCA1-defective subject, a PARP1 inhibitor-resistant subject and a DNA-damaging anti-cancer agent resistant subject having breast cancer or ovarian cancer. The subject may be a BRCA1-defective subject, a PARP1 inhibitor-resistant subject and a DNA-damaging anti-cancer agent resistant subject having breast cancer. The subject may be a BRCA1-defective subject, a PARP1 inhibitor-resistant subject and a DNA-damaging anti-cancer agent resistant subject having ovarian cancer.

In embodiments, the cancer subject is a breast cancer subject and at least one of a BRCA1-defective subject, a PARP1 inhibitor-resistant subject, or a DNA-damaging anti-cancer agent resistant subject. Thus in embodiments, the breast cancer subject is also a BRCA1-defective subject. In embodiments, the breast cancer subject is also a PARP1 inhibitor-resistant subject. In embodiments, the breast cancer subject is also a DNA-damaging anti-cancer agent resistant subject. The breast cancer subject may be a BRCA1-defective subject and a PARP1 inhibitor-resistant subject (e.g. the breast cancer subject has a cancer that has a BRCA1-defect and is resistant to a PARP1 inhibitor). The breast cancer subject may be a BRCA1-defective subject and a DNA-damaging anti-cancer agent resistant subject (e.g. the breast cancer subject has a cancer that has a BRCA1-defect and is resistant to a DNA-damaging anti-cancer agent). The breast cancer subject may be a PARP1 inhibitor-resistant subject and a DNA-damaging anti-cancer agent resistant subject (e.g. the breast cancer subject has a cancer that has resistant to a PARP1 inhibitor and to a DNA-damaging anti-cancer agent). The breast cancer subject may be a BRCA1-defective subject, a PARP1 inhibitor-resistant subject and a DNA-damaging anti-cancer agent resistant subject (e.g. the breast cancer subject has a cancer that has BRCA1-defect and is resistant to a PARP1 inhibitor and a DNA-damaging anti-cancer agent).

In embodiments, the cancer subject is an ovarian cancer subject and at least one of a BRCA1-defective subject, a PARP1 inhibitor-resistant subject, or a DNA-damaging anti-cancer agent resistant subject. Thus in embodiments, the ovarian cancer subject is also a BRCA1-defective subject. In embodiments, the ovarian cancer subject is also a PARP1 inhibitor-resistant subject. In embodiments, the ovarian cancer subject is also a DNA-damaging anti-cancer agent resistant subject. The ovarian cancer subject may be a BRCA1-defective subject and a PARP1 inhibitor-resistant subject (e.g. the ovarian cancer subject has a cancer that has a BRCA1-defect and is resistant to a PARP1 inhibitor). The ovarian cancer subject may be a BRCA1-defective subject and a DNA-damaging anti-cancer agent resistant subject (e.g. the ovarian cancer subject has a cancer that has a BRCA1-defect and is resistant to a DNA-damaging anti-cancer agent). The ovarian cancer subject may be a PARP1 inhibitor-resistant subject and a DNA-damaging anti-cancer agent resistant subject (e.g. the ovarian cancer subject has a cancer that has resistant to a PARP1 inhibitor and to a DNA-damaging anti-cancer agent). The ovarian cancer subject may be a BRCA1-defective subject, a PARP1 inhibitor-resistant subject and a DNA-damaging anti-cancer agent resistant subject (e.g. the ovarian cancer subject has a cancer that has BRCA1-defect and is resistant to a PARP1 inhibitor and a DNA-damaging anti-cancer agent).

In embodiments, the cancer subject is a breast cancer subject, ovarian cancer subject, colon cancer subject, liver cancer subject, kidney cancer subject, lung cancer subject, non-small cell lung cancer subject, brain cancer subject, prostate cancer subject, pancreatic cancer subject, melanoma subject, leukemia subject, or sarcoma subject. In embodiments, the cancer subject is a breast cancer subject or an ovarian cancer subject. In embodiments, the cancer subject is a breast cancer subject. In embodiments, the cancer subject is an ovarian cancer subject.

The subject may have a cancer as described herein, where the cancer exhibits at least one of a BRCA1-defect, resistance to a PARP1 inhibitor, or resistance to a DNA-damaging anti-cancer agent. The cancer may be breast cancer, ovarian cancer, colon cancer, liver cancer, kidney cancer, lung cancer, non-small cell lung cancer, brain cancer, prostate cancer, pancreatic cancer, melanoma, leukemia, or sarcoma. The cancer may be one of the aforementioned cancers having a BRCA1-defect. The may be one of the aforementioned cancers having resistance to a PARP1 inhibitor. The cancer may be one of the aforementioned cancers having resistance to a DNA-damaging anti-cancer agent.

In embodiments, the cancer has a BRCA1-defect and at least one of resistance to a PARP1 inhibitor or a DNA-damaging anti-cancer agent. In embodiments, the cancer has resistance to a PARP1 inhibitor and has at least one of a BRCA1-defect or resistance to a DNA-damaging anti-cancer agent. In embodiments, the cancer has resistant to a DNA-damaging anti-cancer agent and has at least one of a BRCA1-defect or resistance to a PARP1 inhibitor.

The cancer may be breast cancer or ovarian cancer. The cancer may be breast cancer. The cancer may be ovarian cancer. The cancer may be colon cancer. The cancer may be liver cancer. The cancer may be kidney cancer. The cancer may be lung cancer or a non-small cell lung cancer. The cancer may be brain cancer. The cancer may be prostate cancer. The cancer may be pancreatic cancer. The cancer may be melanoma. The cancer may be leukemia. The cancer may be sarcoma.

In embodiments, the administration of COH29 lowers a specific protein's activity or expression in a cancer subject (e.g. a BRCA1-defective subject, a PARP1 inhibitor-resistant subject or a DNA-damaging anti-cancer agent resistant subject). The inhibition may result from the binding of COH-29 to a target protein which may induce the protein's degradation through proteasome recruitment. The change in protein level may, in turn, modulate the expression pattern of the corresponding gene. In embodiments, COH29, inhibits activity or expression of PARP1, Rad51, or BRCA1 in the subject. Analysis may be performed (e.g. microarray analysis) to identify genes that are differentially expressed as a result of COH29 treatment. Accordingly, administering COH29 may lower BRCA1 protein activity or expression in the subject. Administering COH29 may lower PARP1 protein activity or expression in the subject. Administering COH29 may lower Rad51 protein activity or expression in the subject. The subject may be a cancer subject as described herein, including embodiments thereof. In embodiments, the cancer subject is breast cancer subject, ovarian cancer subject, colon cancer subject, liver cancer subject, kidney cancer subject, lung cancer subject, non-small cell lung cancer subject, brain cancer subject, prostate cancer subject, or pancreatic cancer subject. The cancer subject may be a breast cancer subject or an ovarian cancer subject.

In embodiments, the RNA expression profile of a COH29 treated BRCA1-defective subject may be compared with that of a COH29 treated cancer subject that is BRCA1+(e.g. intact BRCA1). Thus in embodiments, COH29 inhibits activity or expression of a protein to a greater extent in a BRCA1-defective subject than in cancer subject that is BRCA1+. Thus, in embodiments, COH29 inhibits PARP1 to a greater extent in a BRCA1-defective subject than in a cancer subject that is BRCA1+. COH29 may inhibit Rad51 to a greater extent in a BRCA1-defective subject than in a cancer subject that is BRCA1+. In embodiments, COH29 treats the BRCA1-defective subject through synthetic lethality. The BRCA1-defective subject is as described herein, including embodiments thereof. In embodiments, the BRCA1-defective subject is also a breast cancer subject or an ovarian cancer subject.

In embodiments, the administration of COH29 lowers a specific protein's activity or expression in a cancer (e.g. a cancer that is BRCA1-defective or resistant to either or both a PARP1 inhibitor or a DNA-damaging anti-cancer agent). The inhibition may result from the binding of COH-29 to a target protein which may induce the protein's degradation through proteasome recruitment. The change in protein level may, in turn, modulate the expression pattern of the corresponding gene. In embodiments, COH29, inhibits activity or expression of PARP1, Rad51, or BRCA1 in the cancer. Analysis may be performed (e.g. microarray analysis) to identify genes that are differentially expressed as a result of COH29 treatment. Thus, administering COH29 may lower BRCA1 protein activity or expression in the cancer. Administering COH29 may lower PARP1 protein activity or expression in the cancer. Administering COH29 may lower Rad51 protein activity or expression in the cancer. The cancer may be a cancer as described herein, including embodiments thereof. In embodiments, the cancer is breast cancer, ovarian cancer, colon cancer, liver cancer, kidney cancer, lung cancer, non-small cell lung cancer, brain cancer, prostate cancer, or pancreatic cancer. The cancer may be breast cancer or ovarian cancer.

In embodiments, the RNA expression profile of a BRCA1-defective cancer treated with COH29 may be compared with that of a BRCA1+ cancer treated with COH29. Thus in embodiments, COH29 inhibits activity or expression of a protein to a greater extent in a cancer that is BRCA1-defective than in a cancer that is BRCA1+. COH29 may inhibit PARP1 to a greater extent in a cancer that is BRCA1-defective than in a cancer that is BRCA1+. COH29 may inhibit Rad51 to a greater extent in a cancer that is BRCA1-defective than in a cancer that is BRCA1+. In embodiments, COH29 treats a cancer that is BRCA1-defective through synthetic lethality. The cancer may be a cancer as described herein, including embodiments thereof. The cancer may be breast cancer or ovarian cancer.

COH29 may exhibit specificity toward BRCA1-defective human cancers through synthetic lethality. Thus, in embodiments, COH29 treats BRCA1-defective subjects, including embodiments thereof. In embodiments, synthetic lethality arises from inhibition of a second protein in the BRCA1-defective cancer. The second protein may be PARP1. The expression profile of a cancer having a BRCA1-defect may be compared to BRCA1+ cancer cells. In embodiments, COH29 decreases PARP1 activity by about 10%, 20%, 30%, 35%, 40%, 45%, 50%, 55%, 60%, 65%, 70%, 75%, 80%, 85%, 90%, 95%, 96%, 97%, 98%, 99%, or 100% in a cancer that is BRCA1-defective. Thus in embodiments, COH29 inhibits PARP1 activity with greater efficacy in BRCA1-defective cancer cells than in BRCA1+ cancer cells. In embodiments, COH29 decreases PARP1 expression by about 5%, 10%, 15%, 20%, 25%, 30%, 40%, 50%, 60%, 70%, 80%, 90%, 95%, or 100% in a cancer that is BRCA1-defective. Thus in embodiments, COH29 inhibits PARP1 expression with greater efficacy in BRCA1-defective cancer cells than in BRCA1+ cancer cells.

The administration of COH29 may inhibit DNA repair in the subject. The administration of COH29 may inhibit base excision repair (BER) (e.g. repair of damaged DNA by, for example, correcting base lesions that arise due to oxidative, alkylation, deamination, and depurinatiation/depyrimidination damage by removing damaged bases using specific glycosylases). The administration of COH29 may inhibit nucleotide excision repair (NER) (e.g. correcting DNA damage resulting in bulky DNA adducts such as damage resulting from UV exposure, by removing a short single stranded DNA segment). The administration of COH29 may inhibit double stranded DNA break repair in the subject (e.g. using the non-homologous end joining (NHEJ pathway), the microhomology mediated end joining (MMEJ) pathway, or by homologous recombination (HR)). The administration of COH29 may inhibit base excision repair, nucleotide excision repair or double stranded DNA break repair in the subject.

In embodiments, the genotoxic profile of COH29, and thus its ability to activate the DNA damage checkpoint and induce DNA damage, may be assessed by detecting modulated activity or expression of proteins such as, for example, ATM, foxo3, γ-H2AX, p53, or Rad51.

The modulation may be an increase in activity or expression or a decrease in activity or expression of a protein. Thus in embodiments, the administration of COH29 increases γ-H2AX activity or expression in the subject. In embodiments, the administration of COH29 increases γ-H2AX activity or expression in the subject. The administration of COH29 may increase γ-H2AX activity or expression in the subject by at least 1-fold, 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, or 20-fold. The increased γ-H2AX activity or expression may indicate activation of the DNA damage checkpoint and induction of DNA damage. In embodiments, the administration of COH29 increases γ-H2AX activity or expression in a cancer as described herein, including embodiments thereof. In embodiments, the administration of COH29 increases γ-H2AX activity or expression in a cancer as described herein, including embodiments thereof. In embodiments, administration of COH29 increases γ-H2AX activity or expression in triple negative breast cancer. Accordingly, in embodiments, administering an effective amount of COH29 treats triple negative breast cancer.

COH29 may inhibit DNA double strand break (DSB) repair. DSBs may be repaired by, for example, homologous recombination (HR) or nonhomologous end joining (NHEJ) pathway. In embodiments, COH29 inhibits HR. In embodiments, COH29 inhibits the NHEJ pathway. The DNA damage response may be prolonged by suppressing the protein level of proteins involved in HR repair, such as for example, BRCA1 and Rad51. In embodiments, the administration of COH29 decreases Rad51 activity or expression in the subject or in a cancer. In embodiments, the administration of COH29 decreases BRCA1 activity or expression in the subject or in a cancer. In embodiments, the expression of BRCA1 or Rad51 is decreased in the subject or in a cancer. In embodiments, the expression of BRCA1 and Rad51 is decreased in the subject or in a cancer.

In another aspect provided herein is a method of treating cancer in a subject in need thereof. The method includes administering COH29 and a DNA-damaging anti-cancer agent in a combined synergistic amount. In embodiments, the subject is as described herein, including embodiments thereof. Thus, in certain embodiments, the subject is a BRCA1 defective subject or a PARP1 inhibitor-resistant subject. The subject may be a BRCA1 defective subject. The subject may be a PARP1 inhibitor-resistant subject.

The cancer may be breast cancer, ovarian cancer, colon cancer, liver cancer, kidney cancer, lung cancer, non-small cell lung cancer, brain cancer, prostate cancer, pancreatic cancer, melanoma, leukemia, or sarcoma. The cancer may be breast cancer or ovarian cancer. Thus, in embodiments, the subject is a breast cancer subject or an ovarian cancer subject. The subject may be a breast cancer subject. The subject may be an ovarian cancer subject. Subjects may also exhibit one or more phenotypes or genotypes as described herein, including embodiments thereof (e.g. a breast cancer subject may also a BRCA1 defective subject or a DNA-damaging anti-cancer agent resistant subject). In embodiments, the subject is a BRCA1-defective subject and a DNA-damaging anti-cancer agent resistant subject. Subjects may have a cancer having resistance to a DNA-damaging anti-cancer agent. The methods herein may afford treatment of cancers having resistance to at least one DNA-damaging anti-cancer agent by co-administering an effective amount of COH29.

In embodiments, the DNA-damaging anti-cancer agent is a chemotherapeutic DNA-damaging agent. The DNA-damaging anti-cancer agent may be an alkylating agent. The DNA-damaging anti-cancer agent may be an antimetabolite as described herein, including embodiments thereof. The DNA-damaging anti-cancer agent may be an anthracycline. The DNA-damaging anti-cancer agent may be a platinum-based agent. The DNA-damaging anti-cancer agent may be a taxane. The DNA-damaging anti-cancer agent may be a kinase inhibitor.

The DNA-damaging anti-cancer agent may be a histone deacetylase inhibitor. The DNA-damaging anti-cancer agent may be a topoisomerase inhibitor. The DNA-damaging anti-cancer agent may be a nucleotide analogue. In embodiments, inhibition of cancer is synergistically increased in the presence of a DNA-damaging cancer agent and COH29.

In embodiments, the method of treating includes inhibiting at least two proteins in synthetic lethality. At least one of the proteins may be BRCA1. At least one of the proteins may be Rad51. At least one of the proteins may be PARP1. In embodiments, the inhibition of PARP1 may be in a BRCA1-defective subject. In embodiments, inhibition of PARP1 is synergistically increased in the presence of a DNA-damaging anti-cancer agent and COH29. The DNA-damaging anti-cancer agent may be gemcitabine, γ-irradiation, or cisplatin, including its derivatives as set forth herein.

The DNA-damaging anti-cancer agent may be cisplatin including its derivatives as described herein. In embodiments, the administration of COH29 increases the cytotoxicity of cisplatin to a level greater than the cytotoxicity of cisplatin when administered alone (e.g. administering COH29 and a cisplatin together in a combined synergistic amount). Cisplatin is a widely used chemotherapeutic whose anticancer activity is mainly attributed to DNA crosslinking in target cells. Thus, in embodiments, the co-administration of COH29 and cisplatin results in a reduction in survivability of cancer cells greater than the reduction in survivability of the cancer cells when either COH29 or cisplatin is administered alone (e.g. administering COH29 and a cisplatin together in a combined synergistic amount).

The DNA-damaging anti-cancer agent may be gemcitabine. In embodiments, the co-administration of COH29 and gemcitabine results in a reduction in survivability of cancer cells greater than the reduction in survivability of the cancer cells when either COH29 or gemcitabine is administered alone (e.g. administering COH29 and a gemcitabine together in a combined synergistic amount). The DNA-damaging anti-cancer agent may be γ-irradiation. In embodiments, the administration of COH29 and treatment with γ-irradiation results in a reduction in survivability of cancer cells greater than the reduction in survivability of the cancer cells when either COH29 or γ-irradiation is administered alone. COH29 may be administered before, during, or after treatment with γ-irradiation.

In another aspect, provided herein is a method of treating cancer in a subject in need thereof. The method includes administering an effective amount of a compound having the structure:

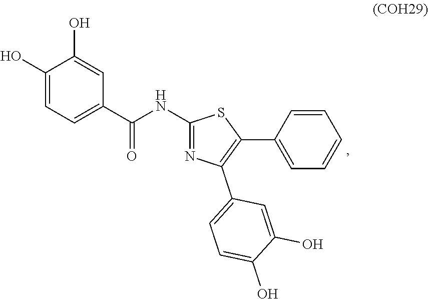 [SMILES: O=C(Nc1nc(-c2ccc(O)c(O)c2)c(-c2ccccc2)s1)c1ccc(O)c(O)c1]

wherein the effective amount is at least about 50 mg per day of administration.

In embodiments, the effective amount is from about 10 mg per day of administration to about 2400 mg per day of administration. In embodiments, the effective amount is from about 100 mg per day of administration to about 2400 mg per day of administration. In embodiments, the effective amount is from about 200 mg per day of administration to about 2400 mg per day of administration. In embodiments, the effective amount is about 100 mg per day of administration. In embodiments, the effective amount is about 200 mg per day of administration. In embodiments, the effective amount is about 300 mg per day of administration. In embodiments, the effective amount is about 400 mg per day of administration. In embodiments, the effective amount is about 500 mg per day of administration. In embodiments, the effective amount is about 600 mg per day of administration. In embodiments, the effective amount is about 700 mg per day of administration. In embodiments, the effective amount is about 800 mg per day of administration. In embodiments, the effective amount is about 900 mg per day of administration. In embodiments, the effective amount is about 1000 mg per day of administration. In embodiments, the effective amount is about 1100 mg per day of administration. In embodiments, the effective amount is about 1200 mg per day of administration. In embodiments, the effective amount is about 1300 mg per day of administration. In embodiments, the effective amount is about 1400 mg per day of administration. In embodiments, the effective amount is about 1500 mg per day of administration. In embodiments, the effective amount is about 1600 mg per day of administration. In embodiments, the effective amount is about 1700 mg per day of administration. In embodiments, the effective amount is about 1800 mg per day of administration. In embodiments, the effective amount is about 1900 mg per day of administration. In embodiments, the effective amount is about 2000 mg per day of administration. In embodiments, the effective amount is about 2100 mg per day of administration. In embodiments, the effective amount is about 2200 mg per day of administration. In embodiments, the effective amount is about 2300 mg per day of administration. In embodiments, the effective amount is about 2400 mg per day of administration. In embodiments, the effective amount is about 2500 mg per day of administration. In embodiments, the effective amount is about 3000 per day of administration. In embodiments, the effective amount is about 3500 per day of administration. In embodiments, the effective amount is about 4000 per day of administration. In embodiments, the effective amount is about 4500 per day of administration. In embodiments, the effective amount is about 5000 per day of administration.

In embodiments, the method includes a course of treatment including administering the compound each day for 21 days followed by 7 days of no administration of the compound. In embodiments, the course of treatment repeated every 28 days. In embodiments, the administering is one time per day. In embodiments, the effective amount is about 100 mg per day of administration or about 200 mg per day of administration. In embodiments, the administering is two times per day. In embodiments, the effective amount is about 300 mg per day of administration or about 400 mg per day of administration. In embodiments, the administering is three times per day. In embodiments, the effective amount is about 600 mg per day of administration. In embodiments, the administering is four times per day. In embodiments, the effective amount is about 800 mg per day of administration. In embodiments, the administering is five times per day. In embodiments, the effective amount is about 1000 mg per day of administration. In embodiments, the administering is six times per day. In embodiments, the effective amount is about 1200 mg per day of administration. In embodiments, the administering is seven times per day. In embodiments, the effective amount is about 1400 mg per day of administration. In embodiments, the administering is eight times per day. In embodiments, the effective amount is about 1600 mg per day of administration. In embodiments, the administering is nine times per day. In embodiments, the effective amount is about 1800 mg per day of administration. In embodiments, the administering is ten times per day. In embodiments, the effective amount is about 2000 mg per day of administration. In embodiments, the administering is eleven times per day. In embodiments, the effective amount is about 2200 mg per day of administration. In embodiments, the administering is twelve times per day. In embodiments, the effective amount is about 2400 mg per day of administration.

Further to any aspect disclosed above for treating cancer in a subject in need thereof or embodiment thereof, in embodiments the subject is a solid tumor cancer subject. In embodiments, the subject is a breast cancer subject or an ovarian cancer subject. In embodiments, the subject is a refractory solid tumor cancer subject. In embodiments, the subject is a breast cancer subject. In embodiments, the breast cancer subject is a BRCA1-defective subject, a PARP1 inhibitor-resistant subject or a DNA-damaging anti-cancer agent resistant subject.

### Pharmaceutical Compositions

In another aspect, there is provided a pharmaceutical composition including a pharmaceutically acceptable excipient and a compound having the structure:

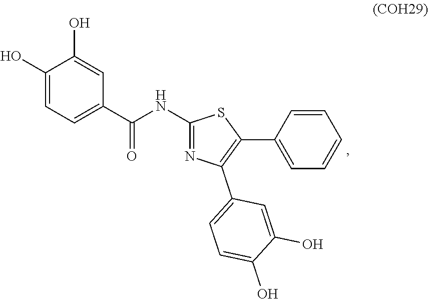 [SMILES: O=C(Nc1nc(-c2ccc(O)c(O)c2)c(-c2ccccc2)s1)c1ccc(O)c(O)c1]

or a pharmaceutically acceptable salt or solvate thereof, wherein the compound is present in an effective amount of at least about 50 mg.

In some embodiments, the effective amount is from about 50 mg to about 2400 mg. In some embodiments, the amount is from about 50 mg to about 2000 mg. In some embodiments, the amount is from about 50 mg to about 1600 mg. In some embodiments, the amount is from about 50 mg to about 1200 mg. In some embodiments, the amount is from about 50 mg to about 800 mg. In some embodiments, the amount is about 50 mg to about 600 mg. In some embodiments, the amount is about 50 mg to about 500 mg. In some embodiments, the amount is about 50 mg to about 400 mg. In some embodiments, the amount is about 50 mg to about 300 mg. In some embodiments, the amount is about 50 mg to about 200 mg. In some embodiments, the amount is about 50 mg to about 100 mg. In some embodiments, the amount is about 50 mg, about 100 mg, about 150 mg, about 200 mg, about 250 mg, about 300 mg, about 350 mg, about 400 mg, about 450 mg, about 500 mg, about 600 mg, about 800 mg, about 1200 mg, about 1600 mg, about 2000 mg, or about 2400 mg.

### Dosing

In one aspect, the COH29 formulations are used for the treatment of diseases and conditions described herein. In addition, a method for treating any of the diseases or conditions described herein in a subject in need of such treatment, involves administration of COH29 formulations in therapeutically effective amounts to said subject.

In some instances, maximum tolerated doses (MTD) and maximum response doses (MRD) for COH29 are determined via established animal and human experimental protocols as well as in the examples described herein. In some instances, toxicity and therapeutic efficacy of COH29 are determined by standard pharmaceutical procedures in cell cultures or experimental animals, including, but not limited to, for determining the LD₅₀ (the dose lethal to 50% of the population) and the ED₅₀ (the dose therapeutically effective in 50% of the population). The dose ratio between the toxic and therapeutic effects is the therapeutic index and it may be expressed as the ratio between LD₅₀ and ED₅₀. COH29 dosages exhibiting high therapeutic indices are of interest. In some instances, the data obtained from cell culture assays and animal studies are used in formulating a range of dosage for use in human. The dosage of COH29 lies preferably within a range of circulating concentrations that include the ED₅₀ with minimal toxicity.

In some embodiments, the pharmaceutical compositions described herein are provided to a subject in need thereof in a dose per day from about 50 mg to about 5000 mg, from about 100 mg to about 2000 mg, from about 200 mg to about 1600 mg, from about 300 mg to about 1200 mg, from about 400 to about 800 mg of COH29, or a pharmaceutically acceptable salt or solvate thereof. In certain embodiments, the COH29 pharmaceutical compositions described herein are provided in a daily dose of about 50 mg, about 51 mg, about 52 mg, about 53 mg, about 54 mg, about 55 mg, about 56 mg, about 57 mg, about 58 mg, about 59 mg, about 60 mg, about 61 mg, about 62 mg, about 63 mg, about 64 mg, about 65 mg, about 66 mg, about 67 mg, about 68 mg, about 69 mg, about 70 mg, about 71 mg, about 72 mg, about 73 mg, about 74 mg, about 75 mg, about 76 mg, about 77 mg, about 78 mg, about 79 mg, about 80 mg, about 81 mg, about 82 mg, about 83 mg, about 84 mg, about 85 mg, about 86 mg, about 87 mg, about 88 mg, about 89 mg, about 90 mg, about 91 mg, about 92 mg, about 93 mg, about 94 mg, about 95 mg, about 96 mg, about 97 mg, about 98 mg, about 99 mg, about 100 mg, about 101 mg, about 102 mg, about 103 mg, about 104 mg, about 105 mg, about 106 mg, about 107 mg, about 108 mg, about 109 mg, about 110 mg, about 111 mg, about 112 mg, about 113 mg, about 114 mg, about 115 mg, about 116 mg, about 117 mg, about 118 mg, about 119 mg, about 120 mg, about 121 mg, about 122 mg, about 123 mg, about 124 mg, about 125 mg, about 126 mg, about 127 mg, about 128 mg, about 129 mg, about 130 mg, about 131 mg, about 132 mg, about 133 mg, about 134 mg, about 135 mg, about 136 mg, about 137 mg, about 138 mg, about 139 mg, about 140 mg, about 141 mg, about 142 mg, about 143 mg, about 144 mg, about 145 mg, about 146 mg, about 147 mg, about 148 mg, about 149 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 610 mg, about 620 mg, about 630 mg, about 640 mg, about 650 mg, about 660 mg, about 670 mg, about 680 mg, about 690 mg, about 700 mg, 710 mg, about 720 mg, about 730 mg, about 740 mg, about 750 mg, about 760 mg, about 770 mg, about 780 mg, about 790 mg, about 800 mg, about 810 mg, about 820 mg, about 830 mg, about 840 mg, about 850 mg, about 860 mg, about 870 mg, about 880 mg, about 890 mg, about 900 mg, 910 mg, about 920 mg, about 930 mg, about 940 mg, about 950 mg, about 960 mg, about 970 mg, about 980 mg, about 990 mg, about 1000 mg, about 1100 mg, about 1200 mg, about 1300 mg, about 1400 mg, about 1500 mg, about 1600 mg, about 1700 mg, about 1800 mg, about 1900 mg, about 2000 mg, about 2100 mg, about 2200 mg, about 2300 mg, or about 2400 mg of COH29, or a pharmaceutically acceptable salt or solvate thereof, or any range derivable therein. The dose per day described herein may be given once per day or multiple times per day in the form of sub-doses given b.i.d., t.i.d., q.i.d., or the like where the number of sub-doses equal the dose per day.

In some embodiments, the compound is administered one time per day. In some embodiments, the compound is administered two times per day. In some embodiments, 100 mg of the compound is administered once per day. In some embodiments, 100 mg of the compound is administered once per day with one 100 mg dose. In some embodiments, 200 mg of the compound is administered twice per day. In some embodiments, 200 mg of the compound is administered twice per day with two 100 mg doses administered about 12 hours apart. In some embodiments, 300 mg of the compound is administered twice per day with one 200 mg dose followed by a 100 mg dose administered about 12 hours later. In some embodiments, 200 mg of the compound is administered in the morning and 100 mg of the compound is administered in the evening. In some embodiments, 300 mg of the compound is administered twice per day. In some embodiments, 400 mg of the compound is administered twice per day. In some embodiments, 400 mg of the compound is administered twice per day with two 200 mg doses administered about 12 hours apart. In some embodiments, 600 mg of the compound is administered per day. In some embodiments, 600 mg of the compound is administered per day with two 300 mg doses administered about 12 hours apart. In embodiments, 800 mg of the compound is administered twice per day with two 400 mg doses administered about 12 hours apart. In some embodiments, 900 mg of the compound is administered twice per day. In some embodiments, 1200 mg of the compound is administered twice per day. In some embodiments, 1200 mg of the compound is administered twice per day. In some embodiments, 1200 mg of the compound is administered twice per day with two 600 mg doses administered about 12 hours apart. In some embodiments, 1600 mg of the compound is administered twice per day. In some embodiments, 1600 mg of the compound is administered twice per day with two 800 mg doses administered about 12 hours apart. In some embodiments, 1800 mg of the compound is administered twice per day with two 900 mg doses administered about 12 hours apart. In some embodiments, 2400 mg of the compound is administered twice per day with two 1200 mg doses administered about 12 hours apart.

In further embodiments, the daily dosages appropriate for the COH29 pharmaceutical compositions are from about 0.5 mg/kg to about 100.0 mg/kg of COH29, or a pharmaceutically acceptable salt or solvate thereof, per body weight. In some embodiments, the daily dosages appropriate for the COH29 pharmaceutical compositions are from about 0.75 mg/kg to about 75 mg/kg of COH29, or a pharmaceutically acceptable salt or solvate thereof, per body weight. In another embodiment, the daily dosage appropriate for the COH29 pharmaceutical compositions are from about 1 mg/kg to about 50 mg/kg of COH29, or a pharmaceutically acceptable salt or solvate thereof, per body weight. In another embodiment, the daily dosage appropriate for the COH29 pharmaceutical compositions is about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, 11 mg/kg, about 12 mg/kg, about 13 mg/kg, about 14 mg/kg, about 15 mg/kg, about 16 mg/kg, about 17 mg/kg, about 18 mg/kg, about 19 mg/kg, about 20 mg/kg, 21 mg/kg, about 22 mg/kg, about 23 mg/kg, about 24 mg/kg, about 25 mg/kg, about 26 mg/kg, about 27 mg/kg, about 28 mg/kg, about 29 mg/kg, about 30 mg/kg, 31 mg/kg, about 32 mg/kg, about 33 mg/kg, about 34 mg/kg, about 35 mg/kg, about 36 mg/kg, about 37 mg/kg, about 38 mg/kg, about 39 mg/kg, about 40 mg/kg, 41 mg/kg, about 42 mg/kg, about 43 mg/kg, about 44 mg/kg, about 45 mg/kg, about 46 mg/kg, about 47 mg/kg, about 48 mg/kg, about 49 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, or about 100 mg/kg of COH29, or a pharmaceutically acceptable salt or solvate thereof.

In some embodiments the COH29 pharmaceutical compositions described herein are provided at the maximum tolerated dose (MTD) for COH29. In other embodiments, the amount of the COH29 pharmaceutical composition administered is from about 10% to about 90% of the maximum tolerated dose (MTD), from about 25% to about 75% of the MTD, or about 50% of the MTD. In some other embodiments, the amount of the COH29 pharmaceutical compositions administered is from about 5%, 10%, 15%, 20%, 25%, 30%, 35%, 40%, 45%, 50%, 55%, 60%, 65%, 70%, 75%, 80%, 85%, 90%, 95%, 99%, or higher, or any range derivable therein, of the MTD for COH29.

In another aspect, there is provided a kit comprising a dispensing apparatus configured to dispense the pharmaceutical composition disclosed herein each day for 21 days followed by 7 days of no administration of the pharmaceutical composition.

In some embodiments, the dispensing apparatus is configured to dispense from 1 to 6 pharmaceutical composition dosage units per day.

In some embodiments, the kit further includes 7 placebo formulation dosage units for administration each day during the 7 days of no administration of said pharmaceutical composition.

### EXAMPLES

The efficacy of DNA-damaging drugs is highly influenced and modulated by cellular DNA repair capacity (9). Indeed, small-molecule inhibitors of DNA repair have been combined with conventional chemotherapy drugs in preclinical studies (18), indicating that the DNA repair machinery is a promising target for novel cancer treatments. Further, PARP inhibitors have been combined with platinum chemotherapy in clinical trials (19, 20). Consistent with these reports, it was discovered, inter alia, that COH29 enhances the sensitivity of cells to cisplatin, especially in BRCA1-deficient cells. This suggests that COH29 synthetic lethality is dependent on NER or BER in HR-deficient cells. COH29 may interfere with several DNA repair pathways (NER, BER, and HR) and may contribute to the cytotoxicity observed in BRCA1-deficient cells in the presence or absence of cisplatin. Thus, COH29 could be exploited as a potent DNA repair inhibitor.

All cell lines were acquired from the American Type Culture Collection (Manassas, Va., USA). Cells were maintained in RPMI 1640 medium (Mediatech) with 10% fetal bovine serum, 2 mM glutamine, and 100 U of penicillin and 100 μg of streptomycin per ml of medium (Sigma) at 37° C. in 5% CO₂. To isolate HCC1937+BRCA1 cells, parental HCC1937 cells were transfected with pcDNA3.1 plasmid expressing full-length BRCA1 cDNA. Stable transfectant clones were selected and used for drug sensitivity assays. For stable transfection, cells at 30-40% confluence were incubated overnight with 2 mg of plasmid DNA, using FUGENE® 6 transfectin reagent (Roche Molecular Biochemical, Monza, Italy) according to the manufacturer's instructions. Cells were then selected in puromycin (1 μg/ml) (Invitrogen Life Technologies, La Jolla, Calif., USA). After 20 to 30 days, viable puromycin-resistant colonies from HCC1937 transfections were expanded and screened. The clones that stably expressed puromycin and retained growth potential were assayed for BRCA1 expression by Western blot analysis. The restoration of BRCA1 expression in the puromycin-resistant cDNA/transfectant cells was evaluated by Western blot analysis. These transfected cells showed an increased expression of BRCA1 protein, suggesting effective restoration of protein expression.

COH29 was synthesized and purified at City of Hope. γ-H2AX was purchased from cell signaling (Danvers, Mass., USA). Rad51 was purchased from Novus (Littleton, Colo., USA). Beta-actin was from Millipore (Billerica, Mass., USA). Antibodies specific to FOXO3 (H-144 and N-16, 1:1000), phospho-H2AX serine-139 (γ-H2AX, 1:1,000), phospho-p53 serine-15 (p53-pS15, 1:1,000), Rad51 (1:1000), β-tubulin (1:1000), Lamin A/C (1:2000 dilution) PARP, and anti-mouse and anti-rabbit IgGs were obtained from Santa Cruz Biotechnology (Santa Cruz, Calif., USA). Abs against FOXO3 (1:1,000) and phospho-ATM serine-1981 (ATM-pS1981, 1:1,000 dilution) were obtained from Epitomics (Burlingame, Calif.) and Millipore (Billerica, Mass.), respectively. An Ab against p53-pS15 was purchased from Cell Signaling Technology (Danvers, Mass.). An anti-p27Kip1 Ab was purchased from BD PharMingen (San Diego, Calif.). Alexa 488 (green)- and Alexa 594 (red)-conjugated secondary Abs were obtained from Molecular Probes (Eugene, Oreg.). Anti-Rabbit IgG (whole molecule)-FITC antibody was purchased from Sigma (St. Louis, Mo., USA). RHODAMINE RED-X™ Goat Anti-Mouse IgG was purchased from Invitrogen (Carlsbad, Calif., USA).

Immunofluorescence experiments were conducted as described previously (21,22). Specifically, A549 cells were grown on glass coverslips. After treatment with COH29 (1 or 10 μM) for 24 or 48 hours, cells were fixed with 4% paraformaldehyde for 10 min and permeabilized with TRITON™ X-100 (0.5%). The coverslips were washed with phosphate-buffered saline (PBS) and blocked with PBS-containing 2% bovine serum albumin (BSA), incubated with an Ab specific to FOXO3 or ATM-pS1981 or γ-H2AX or p53-pS15 (1:50-1:200 dilution), followed by Alexa 488-conjugated anti-rabbit or anti-mouse (1:200), Alexa 594-conjugated anti-goat (1:100) secondary Abs (Molecular Probes). Cells were incubated with 4′,6-diamidino-2-phenylindole (DAPI; Sigma) to stain the nuclei. Specific staining was visualized and images were captured with a Leica SP2 AOBS confocal laser scanning microscope. To measure foci-positive cells, ˜300 cells were used randomly captured by confocal microscopy. The percentages of considering foci-positive cells were calculated from cells containing at least five foci. Each error bar presented is the mean of standard deviation.

For subcellular fractionation, cells were trypsinized and washed with cold PBS solution twice. After centrifugation at 1,200 g for 5 min, cells were incubated in buffer (50 mM HEPES, pH 7.5, 150 mM NaCl, 1 mM EDTA) containing 0.2% NONIDET™ P-40 (NP-40), supplemented with protease inhibitors (5 μg/ml each of pepstatin, leupeptin, and aprotinin) and phosphatase inhibitors on ice for 5 min. Following centrifugation at 1,000 g for 5 min, the supernatant was collected (i.e., cytoplasmic fraction) and pellets were washed with the same buffer twice. The washed samples were extracted for 40 min on ice with fractionation buffer containing 0.5% NP-40 for nuclear fraction. All the samples were sonicated and clarified by centrifugation at 16,000 g for 15 min. Protein concentrations of all fractions were determined with Bio-Rad Protein Assay (Bio-Rad Laboratories, Hercules, Calif.). Immunoblotting was performed as described previously (21,22). Briefly, equal amounts of boiled protein samples were subjected to SDS-polyacrylamide gel electrophoresis (PAGE) and transferred onto nitrocellulose membranes (Bio-Rad Laboratories). Membranes were blocked for 1 hour in 3% BSA in Tris buffered saline containing 0.05% Tween 20 (TBST) and incubated for 1 hour with primary antibody (1:500 or 1:1000) diluted in TBST containing 1% BSA. After two washes with TBST, membranes were incubated for 1 hour with horseradish peroxidase-conjugated secondary Abs (1:3000 dilution) at room temperature. The immunoblots were visualized on film with the West-Q chemi-luminescence kit (GenDEPOT, Barker, Tex.).

The MTT cytotoxicity assay was performed by incubating with MTT and monitoring the MTT formazan formed by viable cells with a microplate reader at a wavelength of 560 nm; the survival ratio was determined using the formula: (Aₜₑₛₜ−A_{blank})/(A_{control}−A_{blank})×100%. The cytotoxicity was determined in 96-well plates using the semiautomatic fluorescence-based Digital Imaging Microscopy System (DIMSCAN). DIMSCAN uses digital imaging microscopy to quantify viable cells, which selectively accumulate FDA (fluorescein diacetate; Alfa Aesar, Ward Hill, Mass.). DIMSCAN is capable of measuring cytotoxicity over a 4 log dynamic range by quantifying total fluorescence per well (which is proportional to the number of viable cells) after elimination of the background fluorescence by digital thresholding and eosin Y (Mallinckrodt Baker, Center Valley, Pa.) quenching. Cells were seeded into 96-well plates in 100 μL of complete medium at 2,000 to 5,000 cells per well, depending on cell line growth rate. After overnight incubation, test compound was added to each well at various concentrations in 50 μL of culture medium. After incubation with the drugs for 96 hours at 37° C., FDA (final concentration: 10 mg/mL) and eosin Y [final concentration: 0.1% (w/v)] were added to each well and the cells were incubated for an additional 20 minutes at 37° C. Total fluorescence per well was then measured using DIMSCAN, and the results were expressed as the ratio of the fluorescence in treated wells to the fluorescence in untreated wells (survival fraction).

Orthotopic Tumor Model. Experiments in mice were conducted under a protocol approved by the IACUC of City of Hope. Because HCC1937 and HCC1937+BRCA1 cells form slow-growing tumors, they were implanted using MATRIGEL™ (Becton-Dickinson Biosciences). To establish tumors, 4×10⁶ cells in 200 μL serum-free medium containing 50% MATRIGEL™ were injected into the mammary fat pads around the inguinal area of a pair of 8 week old female NSG mice. Once the initial tumors reached 13 mm in diameter, they were dissected out, minced into 3 mm pieces and implanted into the inguinal area of the mammary fat pads of the experimental mice. Tumors were measured over a 28-day period, and for each time point, the student t-test was used to determine the statistical significance between daily gavage with 400 mg/kg COH29 in 30% solutol and corresponding vehicle control. Thep value less than 0.05 (2 sides) was considered to indicate statistical significance

The EJ2 cells were generated to evaluate Alt-NHEJ through monitoring the fluorescence intensity of GFP, and EJ5 cells were used to determine NJEJ, as described previously (23). Cells were seeded into 6-well plate and treated with COH29 or cisplatin at different concentration for 24 hours. The cells were then trypsinized, washed, and analyzed by flow cytometry.

The construction of the anti-human BRCA1 siRNA-expressing plasmid was performed as previously described (24). Thus, previously published anti-human BRCA1 siRNA sequences were utilized (5′-UCACAGUGUCCUUUAUGUA-3″ [SEQ ID NO:1] and 5′-UACAUAAAGGACACUGUGA-3′ [SEQ ID NO:2]). In each case, the annealed oligonucleotide duplex encoding the siRNA was subcloned into the expression vector psiRNA-hHlzeo (InvivoGen, San Diego, Calif., USA) to express under the control of the RNA polymerase III-dependent H1 RNA promoter. Cells were transfected with the indicated plasmid at equimolar concentration via electroporation.

Total RNA was isolated using RNEASY® Micro Kit (Qiagen Inc.). Genomic DNA contamination was removed with DNAse I treatment. The integrity of isolated RNA was examined via electrophoresis through 1% agarose gel (SeaKem, FMC, Rockland, Me., USA) or with an Agilent 2100 Bioanalyzer (Agilent Technologies, Santa Clara, Calif., USA). The RNA concentration (A260/A280 ratio) was determined by UV spectrophotometry. The cDNA was prepared from total RNA using MMLV reverse transcriptase and random hexamers as primers (Invitrogen). Gene expression was quantified using cDNA samples through real-time PCR. Primers for BRCA1 were purchased from APPLIED BIOSYSTEMS®, Foster City, Calif., USA. Additional primers and probes for 18S and β-actin were designed according to the APPLIED BIOSYSTEMS® guidelines (PRIMER EXPRESS® software; APPLIED BIOSYSTEMS®) to fit the real-time PCR requirements. The sequences of primers were

(SEQ ID NO: 3)
AGGAATTGCGGGAGGAAAATGGGT
and
(SEQ ID NO:4)
GCCCCCTGAAGATCTTTCTGTCCT.

The PARP1 activity was determined using the PARP1 Chemiluminescent Assay Kit (BPS Bioscience, San Diego) according to the manufacturer's protocol. Briefly, the ribosylation reaction was carried out with activated DNA in PARP assay buffer using test inhibitor, positive control, substrate control and blank reactions, for 1 hour at 25° C. Detection was by streptavidin-HRP with chemiluminescent substrate A and B read in a luminometer. NZebrafish (Danio rerio) were obtained from zebrafish Core facility of Taipei Medical University and maintained at 28° C. on a 14 h light/10 h dark cycle. Embryos were incubated at 28° C. and different developmental stages were determined as described (25). Wild-type embryos were treated with different concentrations of HU (0, 5, 10, 20, 50 mM) or COH29 (0, 10, 20, 50, 100 μM) at 20 hpf to evaluate the mutagenic effect. Fifteen embryos were treated per well condition. Treated embryos were observed at 2, 3, 4, 5 and 6 dpf. At 6 dpf, the percentage of fish exhibiting developmental abnormalities and the survival rate was determined. Embryos were observed using an Olympus IX70-FLA inverted fluorescence microscope. Images were taken using SPOT digital camera system (Diagnostic Instruments, Sterling Heights, Mich., USA) and assembled with Image) software (26).

Microarray samples were RMA normalized (27) using PARTEK® GENOMICS SUITE™ (Version 6.6; Partek, Inc.), and genes were defined as differentially expressed if they showed at least a 1.2 fold-change and false discovery rate (FDR)<0.05. FDR values were calculated using the method of Benjamini and Hochberg (28) from the distribution of ANOVA with Linear Contrastp-values. Gene ontology (GO) (29) enrichment analysis was performed within PARTEK® GENOMICS SUITE™, and GO categories were defined significant with a Fisher Exact test p-value<0.05.

RRM2-PARP1 correlation analysis was determined from gene expression profiling of 289 paraffin embedded breast cancer tumor samples using AFFYMETRIX® U133 A&B (GSE4922) based on the Ivshina et al. study. (30) Statistical analysis was performed using Bioconductor R package, 64 bit, v 3.0.2. (31) Correlation analysis was conducted using Spearman's rank correlation. A level of P<0.05 and r>0.5 was considered statistically significant.

The in vitro replication of the pSVO+ plasmid containing the SV40 replication origin was carried out as previously published (26) with modifications. The final 25 μL reaction volume contained 30 mM HEPES (pH=7.2), 7 mM MgCl₂, 0.5 mM DTT, 5 [α-³²P]-dCTP, 1 μM dCTP, 100 μM each of dTTP, dCTP, and dGTP, 200 μM each of CTP, UTP, and GTP, 4 mM ATP, 40 mM of phosphocreatine, 50 μg of creatine phosphokinase, 50 ng of pSVO+, 0.1-1.0 μg T Ag (optimal concentration determined by titration assays), and optimal amount of HeLa extract (determined by titration assays) (Chimerx; Milwaukee, Wis.). To quantify DNA replication inhibition, HeLa extract was incubated with increasing concentrations of COH29 for 30 minutes prior to the start of the reaction. The HeLa-compound mixture was added to the remaining SV40 DNA replication components, incubated at 37° C. for 1 hour, spotted on WHATMAN® DE81 filters, washed with 100 mM sodium pyrophosphate (pH 7.4) and 300 mM ammonium formate (pH 7.4), then dried. The amount of radiolabeled material incorporated into newly synthesized daughter DNA strands was then determined by liquid scintillation counting.

Without being bound by any particular theory, COH29 anticancer activity may stem at least in part from the inhibition of human ribonucleotide reductase (hRR), which is an enzyme for the biosynthesis of deoxyribonucleotides for DNA replication. In addition, as a component of the base excision repair complex, ribonucleotide reductase is also involved in DNA repair. Consequently, in embodiments, COH29 was discovered herein to target several additional components of the repair complex. Furthermore, in embodiments, BRCA1-defective human breast or ovarian cancer cells are more sensitive than wild-type BRCA1 counterparts to COH29. In embodiments, COH29 exhibits synergy with the DNA crosslinking drugs, such as cisplatin, in BRCA1 mutant cells. In embodiments, COH29 was discovered herein to suppress RAD51, which, without being bound by any particular theory, is involved in the repair of double strand breaks (DSB) by the homologous recombination (HR) pathway. In embodiments, COH29 targets multiple DNA repair pathways and potentially modulates backup DNA repair resulting from the genetic background (mutation). In embodiments, COH29 may overcome acquired resistance to PARP inhibitors (e.g. PARP1 inhibitors). Pharmacologically, and without being bound by any particular theory, COH29 was discovered herein to suppress gemcitabine-resistant human cancer cell proliferation and synergizes with cisplatin or γ-irradiation.

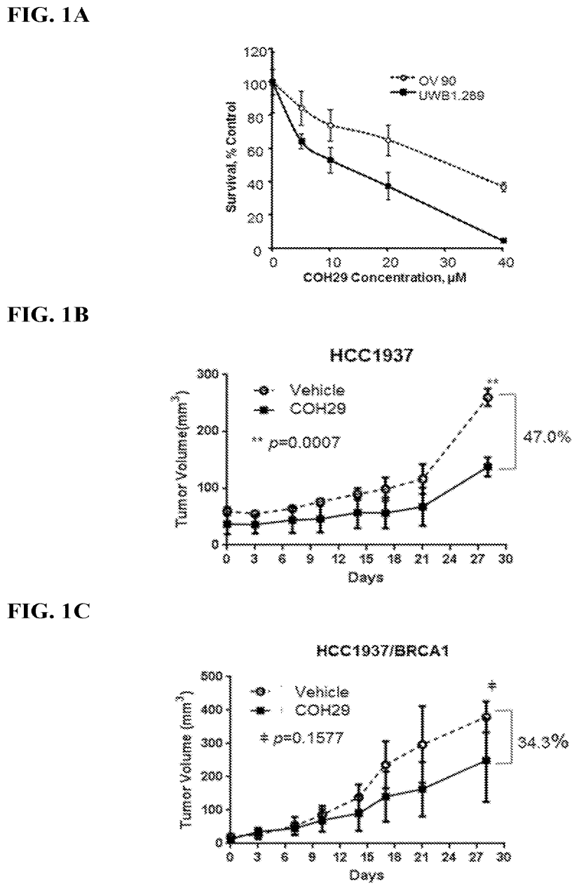

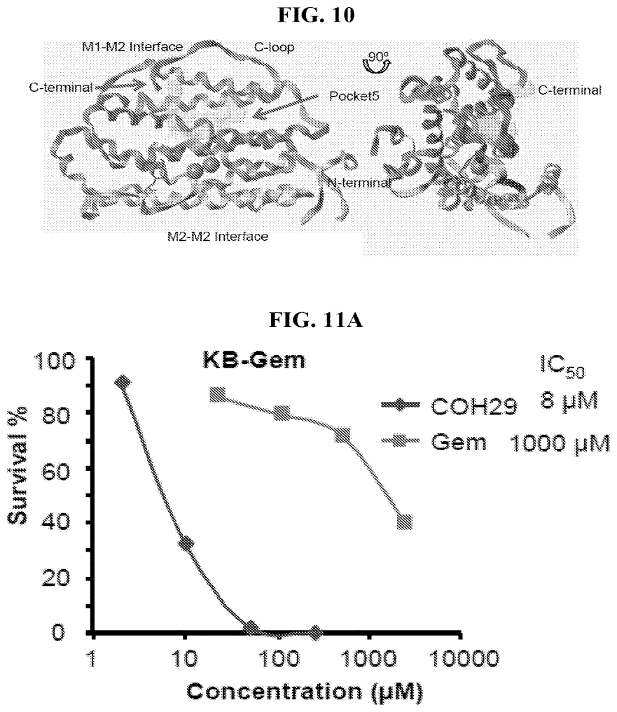

COH29 is an aromatically substituted thiazole compound that, without being bound by any particular theory, occupies a structurally conserved ligand-binding pocket on the hRRM2 subunit located at the hRRM1/hRRM2 interface (FIG. 10). In embodiments, binding to this pocket inhibits the hRRM1/hRRM2 assembly, effectively inhibiting RR activity. In vitro COH29 is active in multiple human cancer cell lines and was shown to be highly potent with an IC₅₀ less than 10 μM in most cases. COH29 has been shown to possess broad activity in the NCI-60 cell line panel, and that multiple human breast cancer cell lines including, for example, human ovarian cancer cell lines, are sensitive to COH29 (6). Breast and ovarian cancers occur with a greater frequency in carriers of a mutant BRCA1 gene than the general population (32). Accordingly, herein, it was investigated whether human cancer cells defective in BRCA1 demonstrated greater sensitivity to COH29. Indeed, as shown in FIG. 1A the UWB1.289 ovarian cancer cell line, which expresses truncated BRCA1 protein due to the homozygous 2594delC mutation (33), was more sensitive to COH29 (IC₅₀: 12.30±1.15 μM) than the OV90 human ovarian cancer cell line that express wild-type BRCA1 (IC₅₀: 31.57±3.35 μM).

The effect of COH29 was assessed in breast cancer cells with an identical genetic background, which differed only in BRCA1 expression to determine the extent that mutant BRCA1 increased cytotoxicity. First, the effect of silencing BRCA1 expression was examined. HCC1937 are human breast cancer cells homozygous for an insertion mutation, resulting in the endogenous expression of a truncated BRCA1 protein (34) and HCC1937+BRCA1 is a stable transfectant clone expressing the human wild-type BRCA1 protein. BRCA1 expression was suppressed by RNA interference in these cells. After 72 h treatment with 10 μM COH29 72% of HCC1937+BRCA1 cells transfected with control siRNA survived. In contrast, only 53% of the cells transfected with BRCA1 siRNA survived. The effect of restoring wild-type BRCA1 expression on COH29 cytotoxicity was investigated by comparing HCC1937 and HCC1937+BRCA1 cells. When treated with varying doses of COH29 for 72 h, cells expressing wild type BRCA1 were much less sensitive to COH29 (IC₅₀: 35.01±3.63 μM) than the BRCA1 mutant HCC1937 cells (IC₅₀: 7.25±0.64 μM). Real-time reverse transcriptase polymerase chain reaction (RT-PCR) showed that HCC1937+BRCA1 cells express ˜2.5 fold higher level of BRCA1 than HCC1937.

The sensitivity of BRCA1 deficient cells to COH29 was further confirmed in an orthotopic tumor explant model. The growth of HCC1937 tumors implanted into mouse mammary fat pads was significantly (p=0.0007) suppressed by daily oral dosing with 400 mg/kg COH29 compared to vehicle (FIG. 1B). In contrast, tumors established with the isogenic HCC1937+BRCA1 cells were not significantly smaller in COH29 treated mice than in vehicle controls (p=0.1577; FIG. 1C).

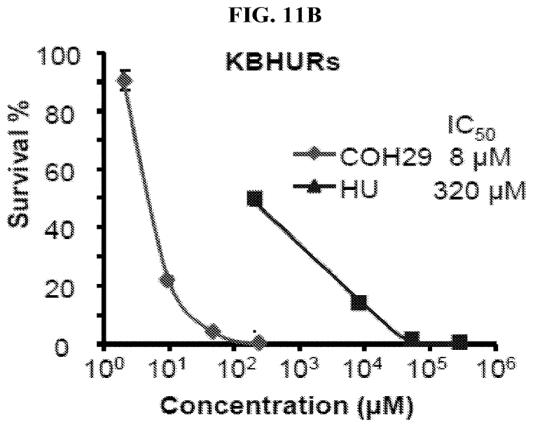

The impact of the BRCA1 mutation on response to COH29 treatment in ovarian cancer cells was also examined. UWB1.289+BRCA1 are a stable transfectant clone of ovarian cancer cells expressing the human wild-type BRCA1 gene, and UWB1.289 are parental cells that were transfected with a control plasmid expressing the neomycin-resistance gene. These cells were treated with varying doses of COH29 for 72 h. Cells expressing wt BRCA1 were less sensitive to COH29 (IC₅₀: 23.52±2.38 μM and 12.30±1.15 μM for UWB1.289+BRCA1 and UWB1.289, respectively). RT-PCR assay showed that UWB1.289+BRCA1 cells express ˜3.08 fold higher level of BRCA1 than UWB1.289. These results suggest that COH29 may induce greater lethality in human cancer cells defective in BRCA1. Additional pharmacological data for COH29 are provided in Tables 1 and 2. Of particular significance is the finding that COH29 suppresses the growth of various human cancer cells resistant to gemcitabine, hydroxyurea or cisplatin (Table 1 and FIGS. 11A (KB-Gem) and 11B (KBHURs)).

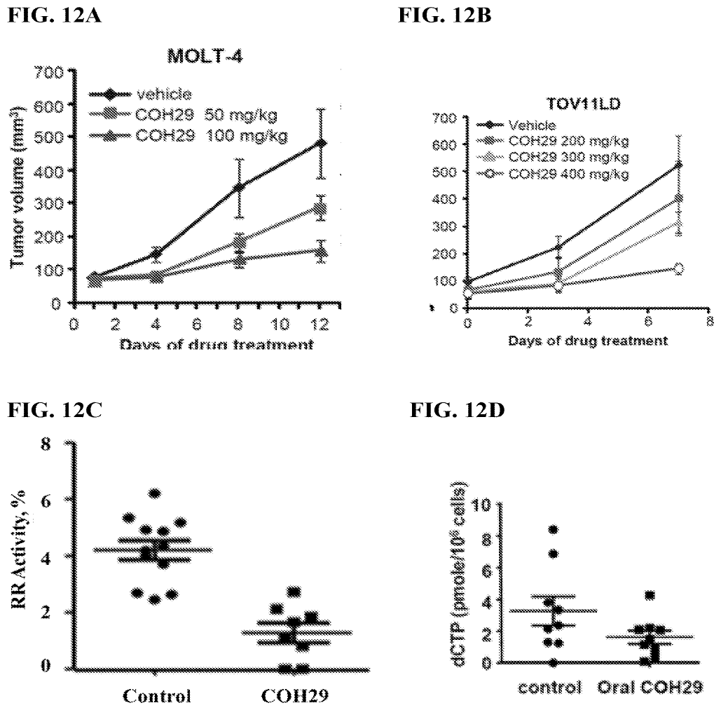

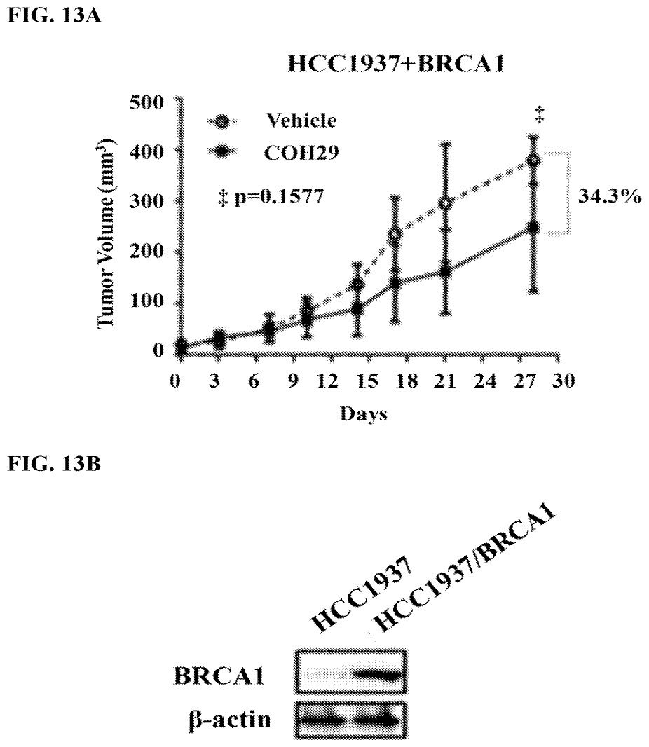

COH29 inhibited lymphoma (MOLT-4) (FIG. 12A) and ovarian (TOV112D) (FIG. 12B) mouse xenograft tumors. The tumor ribonucleotide reductase activity was also reduced with corresponding decrease of intratumoral dNTF pools (FIGS. 12C and 12D). COH29 was shown to be significantly more effective in HCC1937 BRCA1-deficient breast cancer cells in vitro (FIG. 13A) and in mouse mammary fat pad orthotopic tumor xenographs (FIG. 13B) than in HCC1937 BRCA1 wild type breast cancer cells.

To identify the mechanism through which COH29 preferentially lyses BRCA1-mutant human cancer cells, genome-wide microarray analysis was performed using the AFFYMETRIX® GENECHIP® microarray platform to identify the gene expression profiles and pathways affected by COH29 treatment. The RNA expression profile of COH29 treated HCC1937 breast cancer cells lacking BRCA1 was compared with that of COH29 treated HCC1937+BRCA1 cells. Both HCC1937-COH29 and HCC1937+BRCA1-COH29 cells showed Gene Ontology (GO) enrichment for DNA repair genes (Table 1a; p-values ranging from 0.0046-0.0069), suggesting that COH29 interferes with DNA repair pathways. For example, DNA ligation involved in DNA repair is more strongly enriched in HCC1937 cells which may relate to the phenotypic effect. In BRCA1 wild-type cells, COH29 induced DNA damage signaling and suppressed BRCA1 and Rad51 expression, suggesting COH29 may inhibit the homologous recombination (HR) pathway to maintain double strand breaks (DSB) induced by COH29-activated DDR (DNA-damage response).

TABLE 1
Comparison of the Effect of COH29 and Various Antineoplastic Treatments

| | | Antineoplastic Treatment IC₅₀, mean ± SEM | | | | |
|---|---|---|---|---|---|---|
| | | COH29 | Gemcitabine | Cisplatin | Paclitaxel | γ-ray |
| Comparison | Cell line | (μM) | (nM) | (μM) | (nM) | (Gy) |
| Human ovarian | OV90 | 31.57 ± 3.35 | 78.27 ± 11.63 | 1.57 ± 0.29 | 9.09 ± 0.41 | >6 |
| cancer cell lines | TOV112D | 16.00 ± 2.08 | 28.35 ± 4.18 | 1.32 ± 0.20 | 6.45 ± 1.05 | 5.30 ± 0.98 |
| | OVCAR-3 | 20.50 ± 0.81 | 118 ± 18.5 | 1.64 ± 0.04 | 2.89 ± 0.07 | — |
| | OVCAR-4 | 11.25 ± 1.51 | 55.15 ± 3.55 | 2.25 ± 0.25 | 5.55 ± 0.05 | — |
| BRCA1-defective vs | UWB1.289 | 12.30 ± 1.15 | 56.48 ± 0.32 | 1.43 ± 0.87 | 13.88 ± 0.70 | — |
| WT in ovarian cancer | UWB1.289 + | 23.52 ± 2.38 | 42.22 ± 0.62 | 7.40 ± 2.02 | 12.96 ± 1.62 | — |
| | BRCA1 | | | | | |
| Gemcitabine-resistant | KB-Gem | 7.5 ± 0.29 | 60.00 ± 10.00 | 0.38 ± 0.03 | 1.95 ± 0.15 | — |
| vs cisplatin-resistant | KB-7D | 8.48 ± 0.27 | 0.69 ± 0.02 | 7.50 ± 0.30 | 5.17 ± 0.17 | — |
| Cisplatin-sensitive vs | A2780 | 4.67 ± 1.64 | 25.51 ± 1.66 | 0.69 ± 2.11 | 3.60 ± 1.67 | — |
| resistant ovarian cells | A2780-DDDP | 6.47 ± 1.41 | 36.30 ± 0.79 | 5.02 ± 1.18 | 11.38 ± 1.49 | — |
| TNBC | MDA-MB-231 | 9.70 ± 1.52 | 17.00 ± 1.33 | 3.74 ± 0.21 | 2.01 ± 0.57 | 3.60 ± 1.22 |

TABLE 1a
Gene Ontology Enrichment of Genes
Downregulated by COH29 Treatment

| | | | HCC1937 + | |
|---|---|---|---|---|
| | | | BRCA1-COH29 | |
| | HCC1937-COH29 | | vs HCC1937 + | |
| | vs HCC1937 | | BRCA1 | |
| | | Genes | | Genes |
| | P-value: | (n) | P-value: | (n) |
| DNA repair | 0.018 | 17 | 1.6 × 10⁻⁵ | 44 |
| DNA ligation involved | 0.00065 | 3 | 0.0066 | 3 |
| in DNA repair | | | | |
| DSB repair | 0.0015 | 7 | 6.9 × 10⁻⁵ | 14 |
| DSB repair via HR | 0.0069 | 5 | 0.0046 | 9 |
| DSB repair via NHEJ | 0.049 | 2 | 0.041 | 3 |

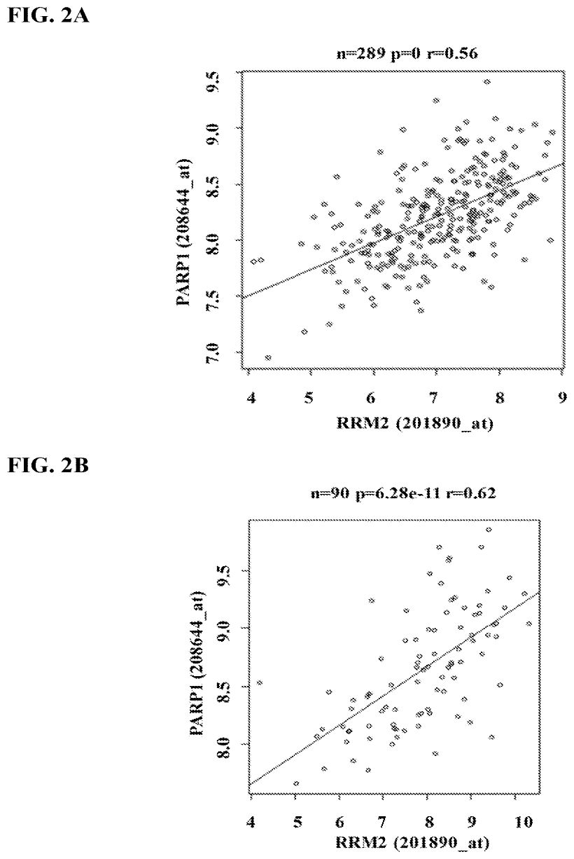

Publicly available gene expression studies in breast and ovarian cancer patient cohorts were examined to verify gene expression correlations between RRM2 and PARP1. A correlation was observed between RRM2 and PARP1 in Ivshina et al. (30) study of breast cancer cohorts (n=289, P=0, r=0.56; FIG. 2A), as well as RRM2 and PARP1 gene expression correlation analysis in the study from Anglesio et al, (35) of ovarian cancer cohorts (n=90, p=0, r=0.62; FIG. 2B). Future genotype-phenotype correlation in selected patient cohorts may help to determine risk profiles for targeted treatments with COH29 in combination with traditional breast and ovarian chemotherapy.

The mechanism through which COH29 preferentially lyses BRCA1-mutant human cancer cells was explored by attempts to identify the target protein. The expression profile of the target protein(s) may be affected through the interaction with COH29. For instance, the binding of COH-29 to a target protein may induce its degradation through proteasome recruitment. The change in protein level may, in turn, alter the expression pattern of the corresponding gene. Microarray analysis was performed to identify the genes that are differentially expressed as a result of COH29 treatment. The RNA expression profile of COH29 treated HCC1937 breast cancer cells lacking BRCA1 was compared with that of COH29 treated HCC1937+BRCA1 cells. The clustering of differentially expressed genes is shown in FIGS. 2A-2B.

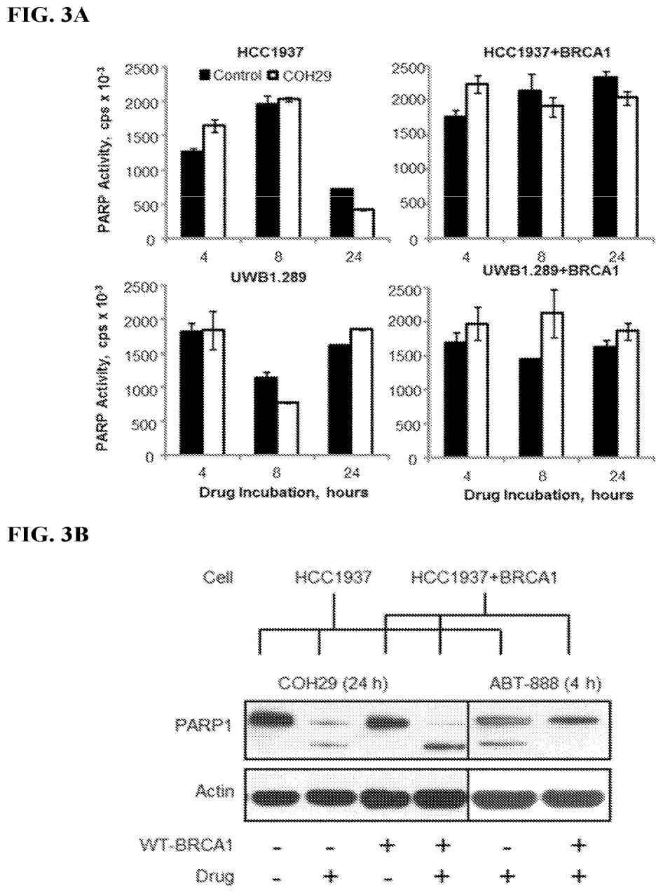

To determine if COH29 inhibited hPARP1, PARP1 activity was examined in lysates of cells treated with or without COH29 for 4 h, 8 h or 24 h. In human breast cancer HCC1937 cells lacking BRCA1 24 h COH29 incubation decreased PARP1 activity by 41.08% (726177 cps for untreated versus 427851 cps for COH29 treated), whereas it decreased by 12.66% (2336878 cps for untreated versus 2041097 cps for COH-29 treated) in similarly treated HCC1937+BRCA1 cells containing BRCA1 (FIG. 3A)

Inhibition of PARP1 by COH29 was more dramatic in the UWB1.289 human ovarian cancer line (FIG. 3A). PARP1 activity decreased by 31.79% (113559 cps for untreated versus 774611 for COH29 treated) in UWB1.289 cells lacking BRCA1 after 8 h COH29 treatment whereas it rose by 46.31% (145769 cps for untreated versus 2129944 cps for COH29 treated) in similarly treated UWB1.289+BRCA1 cells expressing wt BRCA1. Taken together, this indicates that COH29 inhibits PARP1 with greater efficacy in BRCA1-defective human cancer cells.

The effect of COH29 on PARP1 protein levels was also examined. Treatment with COH29 for 24 h attenuated PARP1 protein in HCC1937 BRCA1-defective breast cancer cells and to a lesser extent in HCC1937-BRCA1 wt cells (FIG. 3B). Little reduction was observed for 4 h COH29 treatment. In contrast, ABT-888 treatment for 4 h led to a significant reduction of PARP1 in HCC1937 cells irrespective of their BRCA1 status (FIG. 3B).

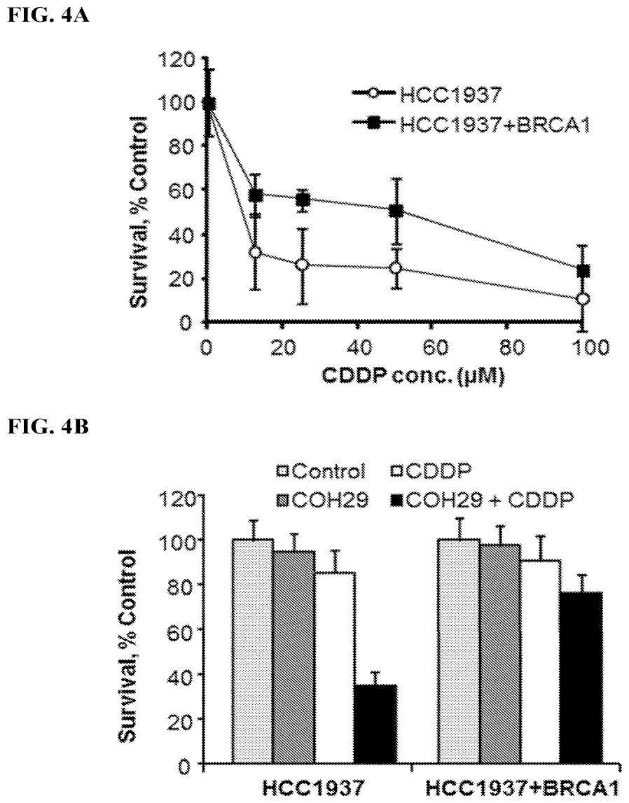

Synthetic lethality achieved through inhibiting PARP1 in a BRCA1-mutant cell background may augment cytotoxicity for DNA-damaging drugs. (18) Whether inhibition of PARP1 by COH29 enhances the cytotoxicity of cisplatin in BRCA1-defective human cancer cells was examined. Cisplatin is a widely used chemotherapeutic whose anticancer activity is mainly attributed to DNA crosslinking in target cells. Stable transfectants of human breast cancer line HCC1937 expressing wt BRCA1 (HCC1937+) or control transfectant (HCC1937) cells were simultaneously treated with COH29 and cisplatin for 24 h. A significant reduction in survivability occurred in HCC1937 cells when compared to HCC1937+BRCA1 cells following treatment with the two drugs (FIG. 4A). Control experiments performed in parallel showed that a single treatment with either COH29 alone or cisplatin alone leads to a lesser yet similar level of impact for the two cell lines (FIG. 4B; also see Table 3). Additional synergy was observed between COH29 and gemcitabine or γ-irradiation (Table 2).

TABLE 2
Synergy of COH29 With Various Antineoplastic Treatments

| | Combination Score | | | | |
|---|---|---|---|---|---|
| | | | MDA-MB- | | |
| Cell line | OV-90 | TOV112D | 231 | A2780 | A2780-CDDP |
| COH29 + | 1.45/Antagonistic | 1.2/0.81 | 3.07/0.93 | 1.09 | 1.08 |
| Cisplatin | | | | | |
| COH29+ | 1.56/Antagonistic | 1.4/1.05 | 1.11/0.87 | 1.06 | 0.96 |
| Gemcitabine | | | | | |
| COH29 + | 1.59/Antagonistic | 1.89 | 1.12/0.91 | ND | ND |
| Radiation | | | | | |

ND; not done

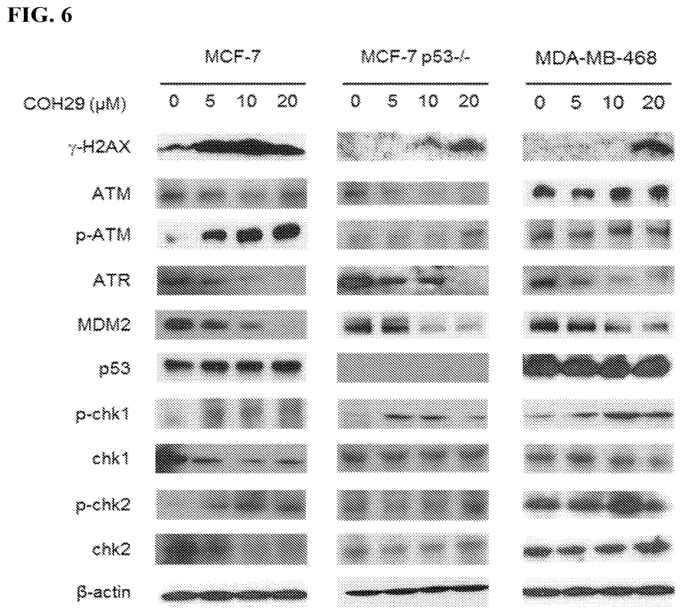

The RR-inhibiting drug hydroxyurea is known to be genotoxic (36, 37). A similar consequence is expected for COH29 as it also inhibits RR. In human cells, such damage activates the DNA damage checkpoint to halt cell cycle progression to allow time for repair. Without being bound by any particular theory, the signaling initiated by the DNA damage is initially mediated by ‘ataxia-telangiectsia-mutated’ (ATM) and ‘ATM and Rad 3-related’ (ATR). Chk1 and Chk2 represent downstream kinases for the signaling event, which phosphorylates Cdc25 phosphatase. Inhibition of Cdc25, in turn, suppresses the Cdk/cyclin complex, resulting in the cell cycle arrest (39). To assess the effect of COH29 treatment on the DNA damage checkpoint, two cell lines that differ in p53 status were employed. COH29 treatment of MCF7 cells containing wild-type p53 activated the DNA damage checkpoint (FIG. 6 left panel) as evidenced by the phosphorylation of ATM. The downstream kinase CHK1 and CHK2 were also phosphorylated. In MCF7 cells lacking p53 (MCF-7 p53−/−), these proteins were also similarly modified following the COH29 treatment (FIG. 6 center panel). Following DNA damage, ATM or ATR phosphorylates γ-H2AX to recruit repair proteins to the site of damaged DNA (39). An increase in the γ-H2AX level occurred following the COH29 treatment in both cell lines. Thus, the COH29 treatment activates the DNA damage checkpoint in a p53-independent manner. Lastly, COH29's impact in ‘triple negative’ human breast cancer cells, which express a reduced level of progesterone receptor, estrogen receptor and Her2 receptor, was examined (33). When MDA-MB-468 cells were treated with COH29, a similar activation profile for the above kinases was observed (FIG. 6, right panel).

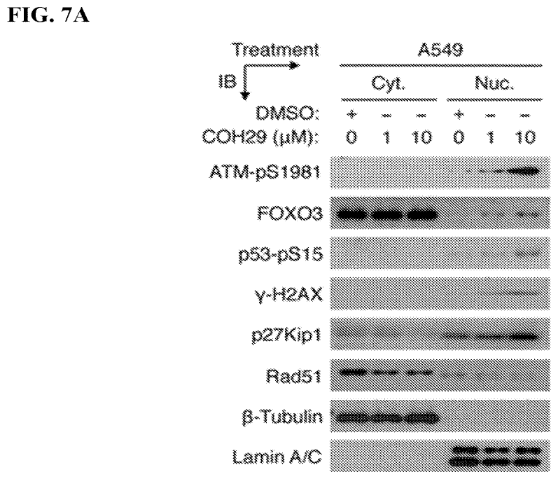

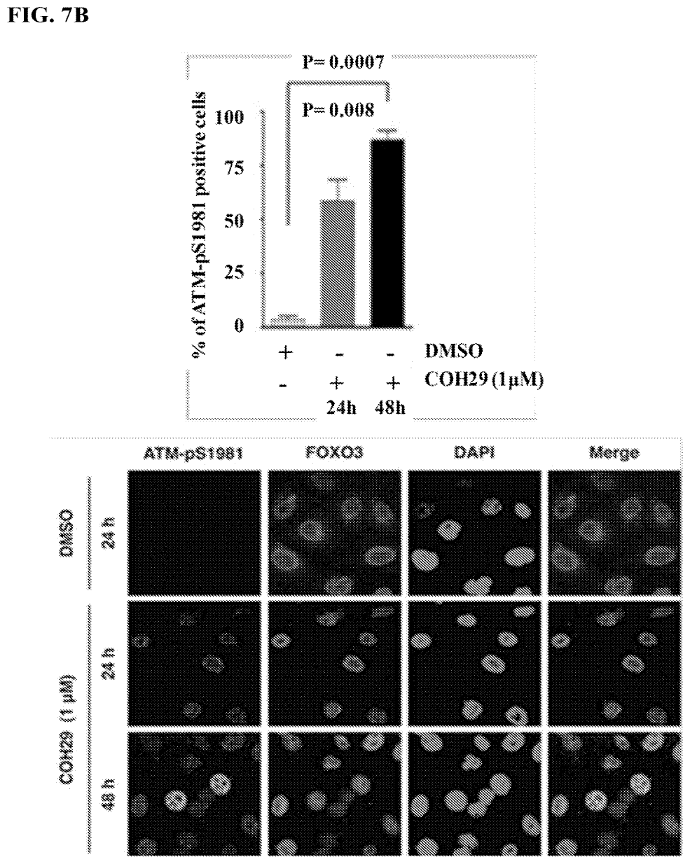

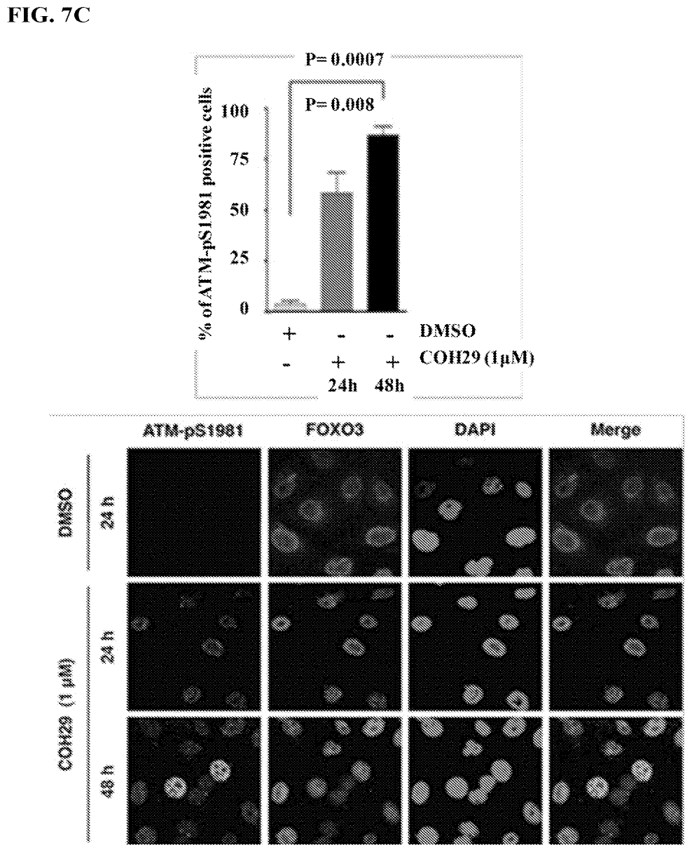

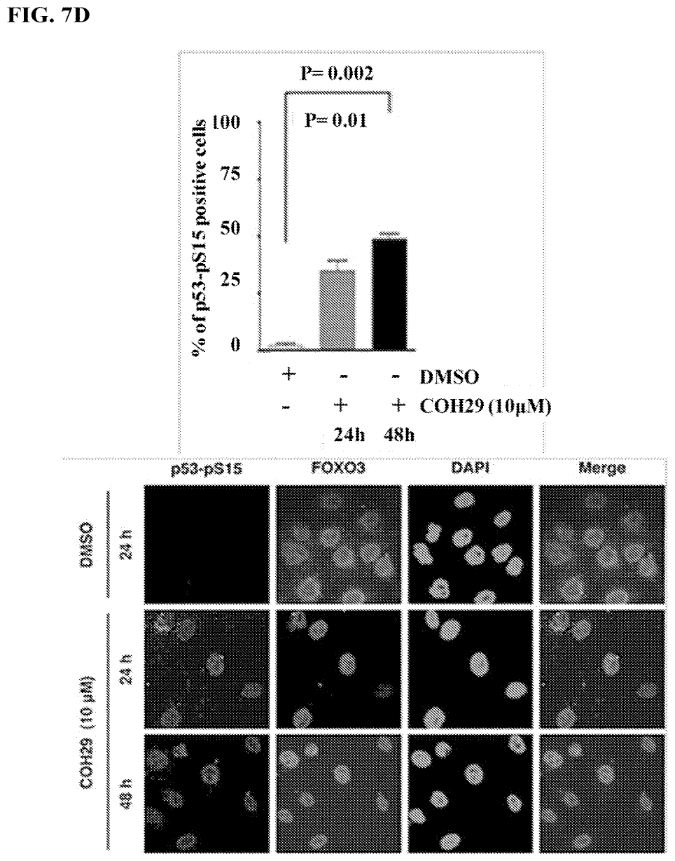

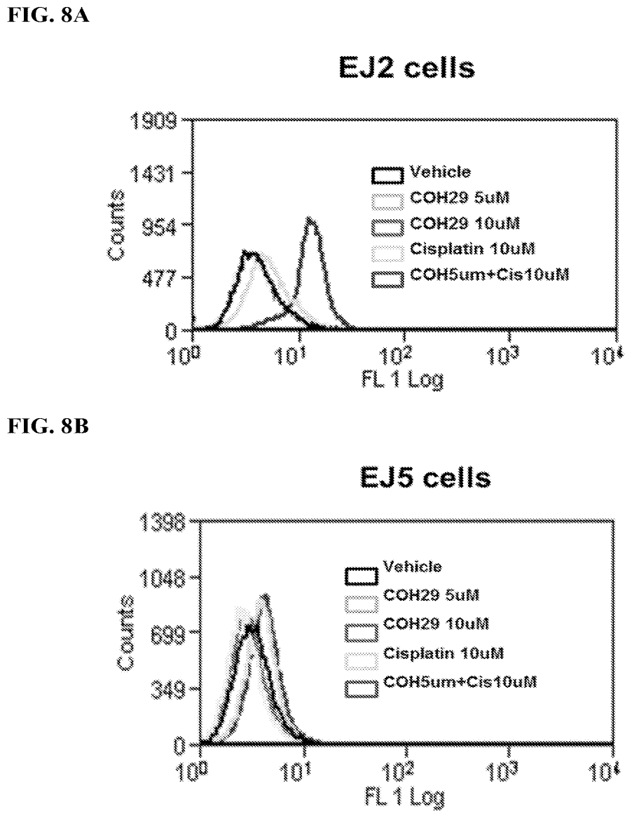

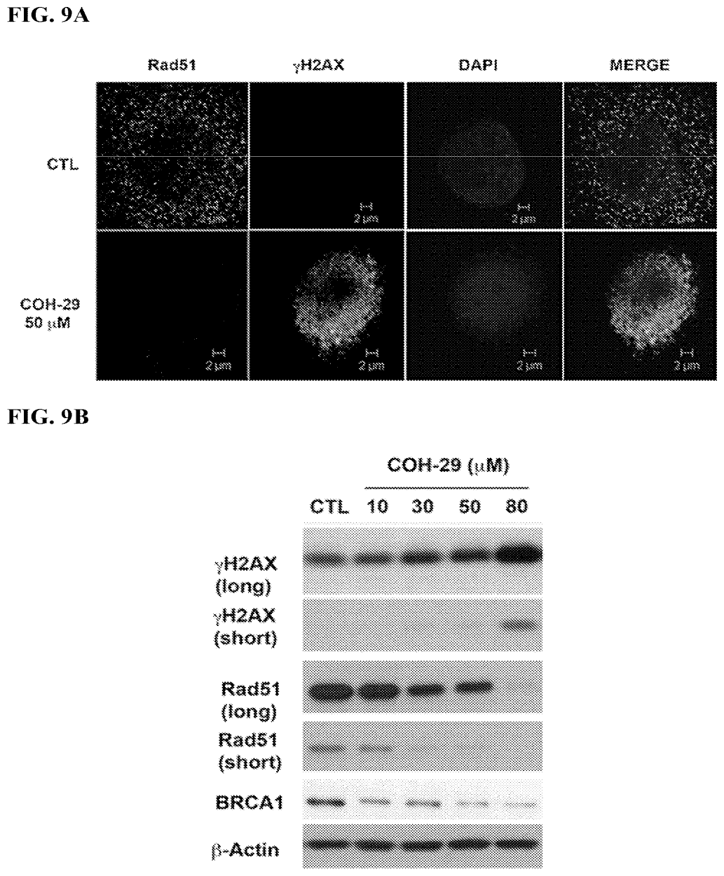

The effect of COH29 in BRCA1 wild-type cells was further evaluated. As shown in FIG. 7A, COH29 also induced accumulation of γ-H2AX, phospho-p53, and phospho-ATM in the nucleus. In addition, the induction of foxo3 and its target protein p27 in the nucleus was observed in COH29-treated cells (FIG. 7A). Moreover, γ-H2AX, phospho-p53, and phospho-ATM were found to colocalize with foxo3 in the nucleus by confocal immunofluorescence microscopy (FIGS. 7B, 7C, and 7D). These results indicate COH29 induces DNA damage as well in BRCA1 wild-type NSCLC A549 cells. DNA double strand break (DSB) can be repaired either by homologous recombination (HR) or nonhomologous end joining (NHEJ) pathway. To further elucidate the role of COH29 in DSB DNA repair, COH29 was determined to have little effect on NHEJ repair efficiency by the GFP-based chromosomal reporter EJ5-GFP in cells (FIGS. 8A-8B). However, the effect of COH29 on expression of crucial protein Rad51 responsible for HR repair was downregulated in the nucleus of BRCA1 wild-type NSCLC A549 cells by Western analysis (FIG. 7A). Furthermore, COH29 suppressed the protein level of BRCA1 and Rad51 foci formation, accompanied with accumulation of the DSB marker γ-H2AX in cells (FIGS. 9A and 9B), suggesting COH29 may be able to prolong DNA damage response (DDR)-induced DSBs by downregulation of the HR pathway in BRCA1 wild-type A549 cells.

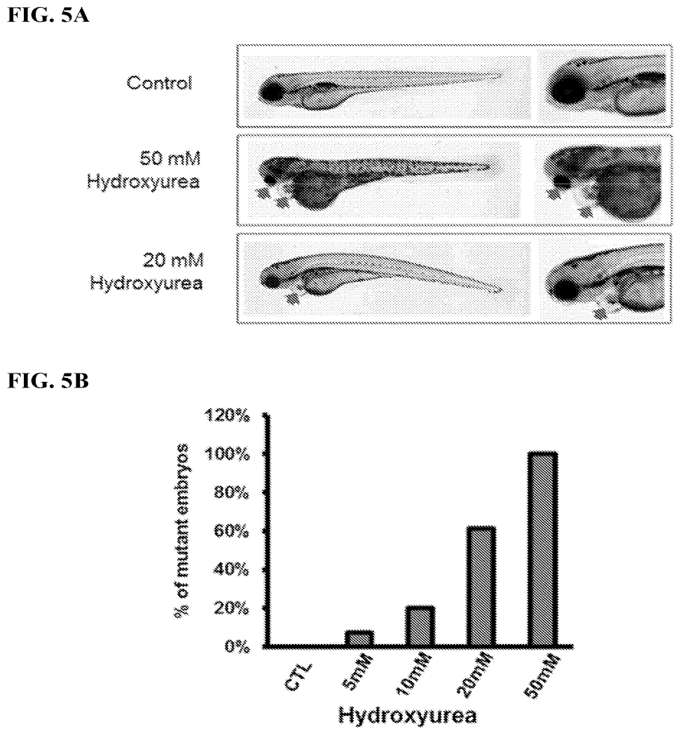

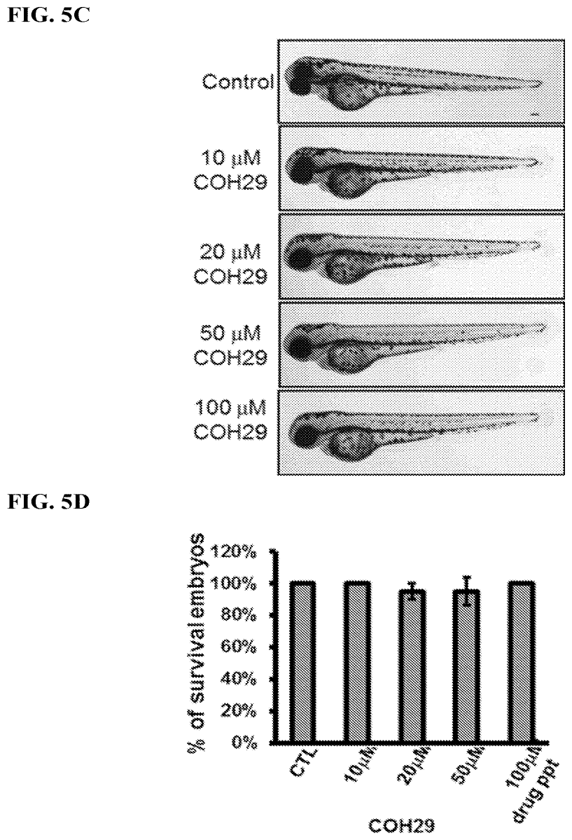

To assess the genotoxicity of COH29, wild-type zebrafish embryos were treated from 1 to 7 dpf (day post-fertilization) with a range of doses of COH29 (0-100 μM) and compared to embryos similarly treated with HU (0-50 mM) which is known to cause developmental defects. As expected, HU caused defects in eyes and heart by 4 dpf (FIG. 5A) and resulted in a dose-dependent increase in the number of mutant embryos (FIG. 5B). In contrast, no developmental defects (FIG. 5C) or decrease in viability (FIG. 5D) were observed in the presence of COH29.

Herein, COH29 was observed to be more active in BRCA1-deficient than in BRCA1 wild-type cell lines, in both in vitro and in vivo studies. BRCA1 is one of the mediators of cellular response to DNA damage. Accordingly, the differential gene expression analysis of COH29 treated BRCA1-deficient and BRCA1 wild-type cells performed herein, identified PARP1 as an additional inhibited protein. COH29 augmented the activity of the DNA damaging agent, cisplatin. COH29 activates the DNA damage checkpoint in p53 independent manner, and that nuclear Rad51 is downregulated.

In human cells, damage to DNA such as the crosslinks may be formed by cisplatin is normally repaired through the BER pathway. As RR provides dNTPs necessary for the repair, the enzyme is closely involved in BER that occurs during S phase. In G1 phase, the p53-inducible subunit p53R2 provides dNTPs for BER. It has been reported that inhibition of RR by COH29 causes dNTP depletion in vivo (6).

COH29 may affect double stranded DNA break repair, as suggested by our data showing suppression of the HR complex protein Rad51. This is indicated by the observation that COH29 causes attenuation in the level of Rad51 protein intracellularly (FIG. 7A). In response to DNA damage, RAD51 translocates from the cytosol to the nucleus to form nucleofilaments on ssDNA, which is an essential step to promote the HR pathway (45,46). In untreated cells, the majority of Rad51 is expressed in the cytosol (FIG. 7B, upper panel). A significantly increased γ-H2AX expression in the nucleus paralleled with a dramatically decreased Rad51 in response to exposure to COH29, suggests Rad51 may play a role in COH29-induced DSBs. This effect of COH29 on Rad51 is similar to that documented for HU, which is known to stall replication forks (47), with the important distinction that COH29 is 20-fold more potent than HU (6), and is not appreciably genotoxic (FIGS. 5A-5D).

In addition, COH29 also suppressed BRCA1, which is another important HR component. It has been reported that inhibition of PARP downregulates BRCA1 and RAD51 expression mediated by E2F4 and p130 (48). Developing inhibitors to interrupt the HR DNA repair machinery (51) has become attractive since elevated Rad51 expression has been observed in numerous types of cancer and is correlated with poor prognosis and drug resistance (52,53). It has been reported that increase of HR capacity by upregulating Rad51 expression level may cause resistance of cancer cells to PARP inhibitors (54). Even in BRCA1-defective cells, loss of 53BP1 can allow partial HR repair and mediate acquired resistance to PARP inhibitors (55). Inactivation of Rad51 functions via downregulating its expression level induced by COH29 may act as a potential therapy for cancers. Our data shows COH29 can interfere with the BER, NER, and HR repair pathways in cells, suggesting COH29 may target backup DNA repair resulting from genetic background or resistance to PARP inhibitors.

Increasing the potency of DNA damaging drugs through synthetic lethality or other means carries the risk of increasing the mutagenic potential of these drugs by suppressing the DNA repair capacity in vivo. In the case of dsB repair pathway, it was shown that inactivation of POLD1 (see above) causes colorectal adenomas and carcinomas (49). Polymorphism of RAD51 is associated with the onset of certain human cancer types (50). Nevertheless, the data herein has showed that COH29 treatment does not appear to render visible morphological anomalies during the embryonic development of zebrafish, unlike HU. The advances described here may lead to further improvement of the current strategy for treating human cancers. The effect of COH-29 on various human breast cancer cells is shown in Table 3.

TABLE 3
Effect of COH29, Cisplatin, and Paclitaxel on Breast Cancer Cell Lines.

| | | COH29 | Cisplatin | Paclitaxel |
|---|---|---|---|---|
| Cell Line | Description | IC₅₀ (μM) | IC₅₀ (μM) | IC₅₀ (mM) |
| HCC1937 | Triple negative | 7.25 ± 0.64 | 2.46 ± 0.11 | 4.04 ± 0.44 |
| HCC1937 + BRCA1 | Triple negative | 35.01 ± 3.63 | 2.93 ± 0.55 | 5.40 ± 0.07 |
| MCF-7 | ER+ PR(+) | 17.61 ± 1.54 | 12.12 ± 1.02 | 6.36 ± 1.11 |
| SKBR3 | ER− PR(−) HER2+ | 13.28 ± 0.03 | 0.99 ± 0.24 | 3.37 ± 0.08 |
| MDA-MB-231 | Triple negative | 9.70 ± 1.52 | 3.74 ± 0.21 | 2.01 ± 0.57 |

### Clinical Study

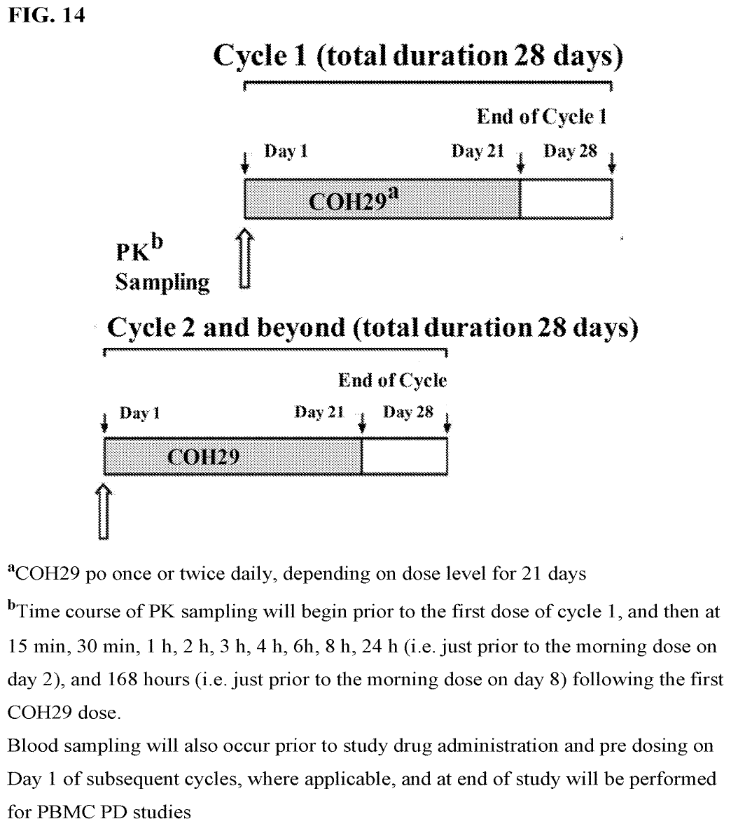

First-in-human Phase I, single site, dose escalation, safety study. Experimental Design Scheme for COH29 is shown in FIG. 14.

Inclusion Criteria: (1) Male and female patients≥18 years of age with advanced, histologically confirmed solid tumors refractory to standard therapy or for which no standard therapy exists; (2) Patients must have measurable or evaluable disease; (3) Patients may be entered if they have received prior radiation therapy involving ≤30% of the bone marrow; (4) Patients may be enrolled with a history of treated brain metastases that are clinically stable for ≥4 weeks prior to enrollment; (5) ECOG Performance Status of ≤2; (6) Life expectancy of greater than 12 weeks as determined by treating clinician assessment; and (7) Adequate organ function per standard laboratory assessments.

Exclusion Criteria: (1) Patients must not have received prior chemotherapy or radiation for ≥4 weeks before study treatment initiation; (2) Patients unable or unwilling to swallow pill; (3) Active heart disease including myocardial infarction within previous 3 months, symptomatic coronary artery disease or heart block, or uncontrolled congestive heart failure; and (4) Women who are pregnant or actively breast-feeding.

Maximum safe starting dose in humans is calculated based on FDA Guidance and based on the following steps: Step 1—Determine the No Observed Adverse Effects Level (NOAEL); Step 2—Human Equivalent Dose Calculation (HED); Step 3—Selection of HED from most appropriate species; and Step 4—Applying a Safety Factor (Standard is to divide by 10). The conversion of animal doses to human equivalent doses based on body surface area in shown in Table 4 below.

TABLE 1
Conversion of Animal Doses to Human Equivalent
Doses Based on Body Surface Area

| | | To Convert Animal | |
|---|---|---|---|
| | To Convert | Dose in mg/kg to | |
| | Animal Dose in | HED^{a} in mg/kg, Either: | |
| | mg/kg to Dose in | Divide | Multiply |
| | mg/m², Multiply | Animal | Animal |
| Species | by kₘ | Dose By | Dose By |
| Human | 37 | — | — |
| Child (20 kg)^{b} | 25 | — | — |
| Mouse | 3 | 12.3 | 0.08 |
| Hamster | 5 | 7.4 | 0.13 |
| Rat | 6 | 6.2 | 0.16 |
| Ferret | 7 | 5.3 | 0.19 |
| Guinea pig | 8 | 4.6 | 0.22 |
| Rabbit | 12 | 3.1 | 0.32 |
| Dog | 20 | 1.8 | 0.54 |
| Primates: | | | |
| Monkeys^{c} | 12 | 3.1 | 0.32 |
| Marmoset | 6 | 6.2 | 0.16 |
| Squirrel monkey | 7 | 5.3 | 0.19 |
| Baboon | 20 | 1.8 | 0.54 |
| Micro-pig | 27 | 1.4 | 0.73 |
| Mini-pig | 35 | 1.1 | 0.95 |

### Preclinical Toxicology

No toxicity concerns were observed in 2 species animal toxicology studies recommended by the FDA. In the pilot study, the lowest dose that was observed to inhibit growth of tumor xenografts was 50 mg/kg by daily oral administration in IL2-rg(KO)/NOD-Scid mice. Toxicity studies in rats and dogs at doses equivalent to 10× that dose have not shown toxicity at 10 or 21 days. These results are shown in Table 5 below.

TABLE 5
COH29 toxicity studies in rats and dogs

| Species | Top dose* | | Duration | Toxicity | Status |
|---|---|---|---|---|---|
| Sprague-Dawley | 125 | mg/kg | 10 days | none | complete |
| Rats (GLP - | | | 21 days | none | complete |
| Charles River | | | | | |
| Beagles (GLP - | 42 | mg/kg | 10 days | none | complete |
| Charles River | | | 21 days | none | complete |

*Dose equivalent to 500 mg/kg/day in mouse (10x the minimum observed for biologic activity)

Dose levels used in the clinical study are shown in Table 6 below. The dose of 280 mg daily is approximately 1/10^{th} of the HED of the 250 mg/kg/day NOAEL in rats (more sensitive species) vs. 84 mg/kg/day in dogs.

TABLE 6
COH29 dose levels

| Dose | COH29 Total | | |
|---|---|---|---|
| Level | Daily Dosage | | Dose |
| −1 | 100 | mg/day | 1 capsule daily |
| 1 | 200 | mg/day | 1 capsule twice daily |
| 2 | 300 | mg/day | 2 capsules AM, |
| | | | 1 capsule PM |
| 3 | 400 | mg/day | 2 capsules twice daily |
| 4 | 600 | mg/day | 3 capsules twice daily |
| 5 | 800 | mg/day | 4 capsules twice daily |
| 6 | 1200 | mg/day | 6 capsules twice daily |
| 7 | 1600 | mg/day | 8 capsules twice daily |
| 8 | 2400 | mg/day | 12 capsules twice daily |
| 9 | 3200 | mg/day | 16 capsules twice daily |
| 10 | 4800 | mg/day | 24 capsules twice daily |

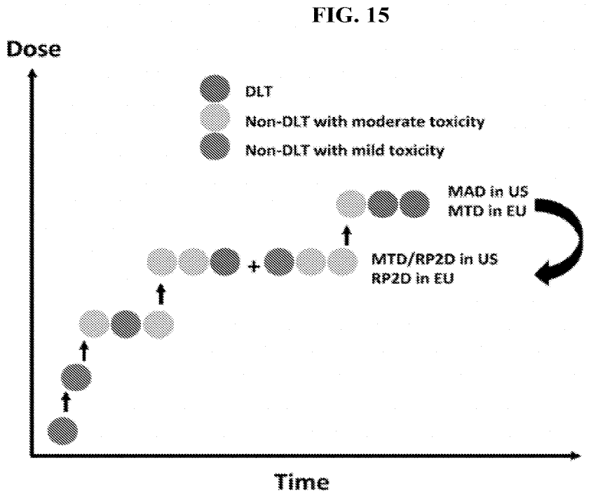

Accelerated titration phase I design is shown in FIG. 15. Intrapatient dose escalation is allowed and design reverts to standard 3+3 if 2 moderate toxicities or 1 dose limiting toxicity (DLT) is encountered.

With respect to the DLT definition, Grade≥3 non-hematological toxicity will be considered dose limiting with the following clarifications: Grade 3 diarrhea will only be considered dose limiting if it is refractory to treatment and unable to be corrected to grade 1 or less within 24 hours. Bloody or grade 4 diarrhea will be dose limiting; Grade 3 nausea and vomiting will only be considered dose limiting if it is refractory to anti-emetic therapy and unable to be corrected to grade 1 or less within 24 hours; Grade 3 rise in creatinine, not corrected to grade 1 or less within 24 hours with IV fluids will be considered dose limiting. All grade 4 rises in creatinine will be dose limiting; and Grade 3 electrolyte toxicities unable to be corrected to grade 1 or baseline within 24 hours will be considered dose limiting. Tumor pain will not be considered dose limiting unless it is refractory to optimal treatment with analgesics. With respect to Grade 4, the following will be considered: Grade 4 thrombocytopenia; Grade 4 neutropenia lasting ≥5 days or febrile neutropenia; Grade 4 hemolysis; Treatment delay>2 weeks as a result of unresolved toxicity; and any degree of anemia, leucopenia in the absence of grade 4 neutropenia lasting ≥5 days, or lymphopenia will not be considered dose limiting.

Maximum dose level is capped at 2400 mg/day (Dose Level 8) corresponding to closest highest level achieved in animal studies. Real time PK monitoring performed to ensure dosing does not exceed AUC of 5000 hr·ng/mL which was maximum level observed in animal toxicology studies. Intrapatient dose escalation is permissible if a dose level has been cleared.

The first patient enrolled into dose level 1 (100 mg twice a day) and cleared without any DLT. The second patient enrolled onto dose level 3 (200 mg twice a day and had moderate toxicity of grade 2 skin rash. The third patient enrolled into dose level 5 (400 mg twice a day and cleared without any DLT. The fourth patient enrolled into dose level 7 (800 mg twice a day and had moderate toxicity of grade 2 diarrhea. The trial now converted to a standard 3+3 dose escalation design. The fifth patient enrolled at dose level 7. The sixth patient is in screening at dose level 7. Realtime PK monitoring was performed for every patient in cycle 1 with no exceeding of AUC safety threshold set by the FDA to date.

Patients receive oral COH29 twice a day for 21 days of a 28-day cycle. Dose levels range from 200 to 2400 mg per day of administration. Dose escalation is pursued utilizing a Simon's accelerated titration design, which allows skipping of dose levels (dose doubling) during the accelerated dose-finding phase. PD assessment includes measurement of plasma CK18 levels to determine degree of cellular apoptosis, evaluation of dNTP pool levels in peripheral blood mononuclear cells (PBMCs) to evaluate RNR inhibition, as well as measurement of PAR expression in PBMCs to assess PARP inhibition. Quantitation of tumor RRM2 expression using dual-color immunohistochemistry is explored as a predictive biomarker of anti-tumor response to COH29.

### NUMBERED EMBODIMENTS

1. A method of treating cancer in a subject in need thereof, said method comprising administering an effective amount of a compound having the structure:

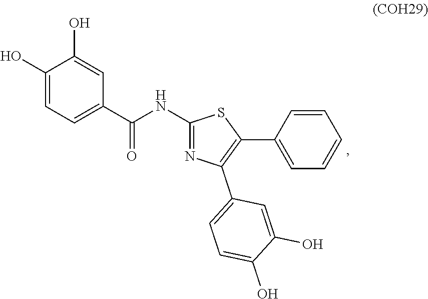 [SMILES: O=C(Nc1nc(-c2ccc(O)c(O)c2)c(-c2ccccc2)s1)c1ccc(O)c(O)c1]

wherein said effective amount is at least about 50 mg per day of administration.

2. The method of embodiment 1, wherein said effective amount is from about 50 mg per day of administration to about 2400 mg per day of administration.

3. The method of embodiment 1 or 2, wherein said effective amount is from about 100 mg per day of administration to about 2400 mg per day of administration.

4. The method of any one of embodiments 1 to 3, wherein said effective amount is from about 200 mg per day of administration to about 2400 mg per day of administration.

5. The method of any one of embodiments 1 to 3, wherein said effective amount is about 100 mg per day of administration.

6. The method of any one of embodiments 1 to 4, wherein said effective amount is about 200 mg per day of administration.

7. The method of any one of embodiments 1 to 4, wherein said effective amount is about 300 mg per day of administration.

8. The method of any one of embodiments 1 to 4, wherein said effective amount is about 400 mg per day of administration.

9. The method of any one of embodiments 1 to 4, wherein said effective amount is about 500 mg per day of administration.

10. The method of any one of embodiments 1 to 4, wherein said effective amount is about 600 mg per day of administration.

11. The method of any one of embodiments 1 to 4, wherein said effective amount is about 700 mg per day of administration.

12. The method of any one of embodiments 1 to 4, wherein said effective amount is about 800 mg per day of administration.

13. The method of any one of embodiments 1 to 4, wherein said effective amount is about 900 mg per day of administration.

14. The method of any one of embodiments 1 to 4, wherein said effective amount is about 1000 mg per day of administration.

15. The method of any one of embodiments 1 to 4, wherein said effective amount is about 1100 mg per day of administration.

16. The method of any one of embodiments 1 to 4, wherein said effective amount is about 1200 mg per day of administration.

17. The method of any one of embodiments 1 to 4, wherein said effective amount is about 1300 mg per day of administration.

18. The method of any one of embodiments 1 to 4, wherein said effective amount is about 1400 mg per day of administration.

19. The method of any one of embodiments 1 to 4, wherein said effective amount is about 1500 mg per day of administration.

20. The method of any one of embodiments 1 to 4, wherein said effective amount is about 1600 mg per day of administration.

21. The method of any one of embodiments 1 to 4, wherein said effective amount is about 1700 mg per day of administration.

22. The method of any one of embodiments 1 to 4, wherein said effective amount is about 1800 mg per day of administration.

23. The method of any one of embodiments 1 to 4, wherein said effective amount is about 1900 mg per day of administration.

24. The method of any one of embodiments 1 to 4, wherein said effective amount is about 2000 mg per day of administration.

25. The method of any one of embodiments 1 to 4, wherein said effective amount is about 2100 mg per day of administration.

26. The method of any one of embodiments 1 to 4, wherein said effective amount is about 2200 mg per day of administration.

27. The method of any one of embodiments 1 to 4, wherein said effective amount is about 2300 mg per day of administration.

28. The method of any one of embodiments 1 to 4, wherein said effective amount is about 2400 mg per day of administration.

29. The method of any one of embodiments 1 to 4, wherein said effective amount is about 2500 mg per day of administration.

30. The method of any one of embodiments 1 to 29, wherein the method comprises a course of treatment comprising administering said compound each day for 21 days followed by 7 days of no administration of said compound.

31. The method of any one of embodiments 1 to 30, wherein said course of treatment repeated every 28 days.

32. The method of any one of embodiments 1 to 31, wherein said administering is one time per day.

33. The method of embodiment 32, wherein said effective amount is about 100 mg per day of administration or about 200 mg per day of administration.

34. The method of any one of embodiments 1 to 31, wherein said administering is two times per day.

35. The method of embodiment 34, wherein said effective amount is about 300 mg per day of administration or about 400 mg per day of administration.

36. The method of any one of embodiments 1 to 31, wherein said administering is three times per day.

37. The method of embodiment 35, wherein said effective amount is about 600 mg per day of administration.

38. The method of any one of embodiments 1 to 31, wherein said administering is four times per day.

39. The method of embodiment 38, wherein said effective amount is about 800 mg per day of administration.

40. The method of any one of embodiments 1 to 31, wherein said administering is five times per day.

41. The method of any one of embodiments 1 to 31, wherein said administering is six times per day.

42. The method of embodiment 41, wherein said effective amount is about 1200 mg per day of administration.

43. The method of any one of embodiments 1 to 31, wherein said administering is seven times per day.

44. The method of any one of embodiments 1 to 31, wherein said administering is eight times per day.

45. The method of embodiment 44, wherein said effective amount is about 1600 mg per day of administration.

46. The method of any one of embodiments 1 to 31, wherein said administering is nine times per day.

47. The method of any one of embodiments 1 to 31, wherein said administering is ten times per day.

48. The method of any one of embodiments 1 to 31, wherein said administering is eleven times per day.

49. The method of any one of embodiments 1 to 31, wherein said administering is twelve times per day.

50. The method of embodiment 49, wherein said effective amount is about 2400 mg per day of administration.

51. The method of one of embodiments 1 to 50, wherein said subject is a solid tumor cancer subject.

52. The method of any one of embodiments 1 to 51, wherein said subject is a breast cancer subject or an ovarian cancer subject.

53. The method of any one of embodiments 1 to 52, wherein said subject is a refractory solid tumor cancer subject.

54. The method of embodiment 51 or 53, wherein said subject is a breast cancer subject.

55. The method of embodiment 54, wherein said breast cancer subject is a BRCA1-defective subject, a PARP1 inhibitor-resistant subject or a DNA-damaging anti-cancer agent resistant subject.

56. The method of any one of embodiments 1 to 55, wherein said administering inhibits DNA repair in said subject.

57. The method of any one of embodiments 1 to 55, wherein said administering inhibits base excision repair (BER), nucleotide excision repair (NER) or double stranded DNA break repair in said subject.

58. The method of any one of embodiments 1 to 55, wherein said administering increases γ-H2AX protein activity or expression in said subject.

59. The method of any one of embodiments 1 to 55, wherein said administering lowers Rad51 protein activity or expression in said subject.

60. The method of one of embodiments 1 to 55, wherein said administering lowers BRCA1 protein activity or expression in said subject.

61. The method of one of embodiments 1 to 55, wherein said administering lowers PARP1 protein activity or expression in said subject.

62. A pharmaceutical composition comprising a pharmaceutically acceptable excipient and a compound having the structure:

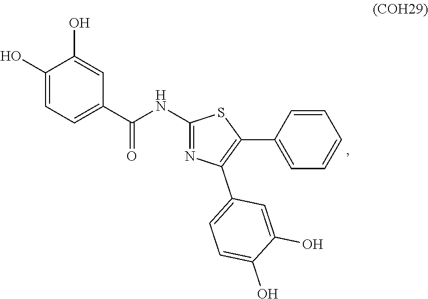 [SMILES: O=C(Nc1nc(-c2ccc(O)c(O)c2)c(-c2ccccc2)s1)c1ccc(O)c(O)c1]

wherein said compound is present in an amount of at least about 50 mg.

63. The pharmaceutical composition of embodiment 62, wherein said effective amount is from about 50 mg to about 1000 mg.

64. The pharmaceutical composition of embodiment 62 or 63, wherein said amount is from about 50 mg to about 500 mg.

65. The pharmaceutical composition of any one of embodiments 62 to 64, wherein said amount is from about 50 mg to about 400 mg.

66. The pharmaceutical composition of any one of embodiments 62 to 65, wherein said amount is from about 50 mg to about 300 mg.

67. The pharmaceutical composition of any one of embodiments 62 to 66, wherein said amount is from about 50 mg to about 200 mg.

68. The pharmaceutical composition of any one of embodiments 62 to 67, wherein said amount is about 100 mg to about 200 mg.

69. The pharmaceutical composition of any one of embodiment 62 to 68, wherein said amount is about 50 mg, about 100 mg, about 150 mg, about 200 mg, about 250 mg, about 300 mg, about 350 mg, about 400 mg, about 450 mg, or about 500 mg.

70. The pharmaceutical composition of any one of embodiments 62 to 69, wherein said pharmaceutical composition is an oral pharmaceutical composition.

71. The pharmaceutical composition of any one of embodiments 62 to 70, wherein said oral pharmaceutical composition is a tablet or capsule.

72. A kit comprising a dispensing apparatus configured to dispense the pharmaceutical composition of one of embodiments 62 to 71 each day for 21 days followed by 7 days of no administration of said pharmaceutical composition.

73. The kit of embodiment 72, wherein said dispensing apparatus is configured to dispense from 1 to 6 pharmaceutical composition dosage units per day.

74. The kit of embodiment 72 or 73, wherein said kit further comprises 7 placebo formulation dosage units for administration each day during the 7 days of no administration of said pharmaceutical composition.

75. The method of any one of embodiments 1 to 50, wherein said subject is a hematological cancer subject.

76. The method of any one of embodiments 1 to 50 and 75, wherein said subject is a leukemia cancer subject.

1. A method of treating cancer in a subject in need thereof, said method comprising administering an effective amount of a compound having the structure: 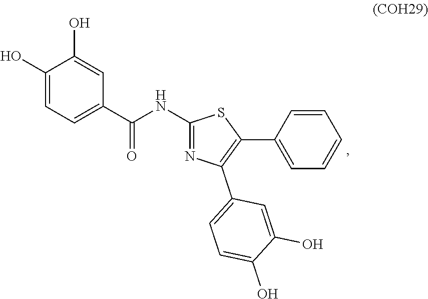 [SMILES: O=C(Nc1nc(-c2ccc(O)c(O)c2)c(-c2ccccc2)s1)c1ccc(O)c(O)c1]
wherein said effective amount is at least about 50 mg per day of administration.

2. The method of claim 1, wherein said effective amount is from about 50 mg per day of administration to about 2400 mg per day of administration.

3. The method of claim 1, wherein the method comprises a course of treatment comprising administering said compound each day for 21 days followed by 7 days of no administration of said compound.

4. The method of claim 3, wherein said course of treatment repeated every 28 days.

5. The method of claim 1, wherein said administering is one time per day.

6. The method of claim 1, wherein said subject is a solid tumor cancer subject.

7. The method of claim 6, wherein said subject is a breast cancer subject or an ovarian cancer subject.

8. The method of claim 1, wherein said subject is a refractory solid tumor cancer subject.

9. The method of claim 6, wherein said subject is a breast cancer subject.

10. The method of claim 9, wherein said breast cancer subject is a BRCA1-defective subject, a PARP1 inhibitor-resistant subject or a DNA-damaging anti-cancer agent resistant subject.

11. The method of claim 1, wherein said administering inhibits DNA repair in said subject.

12. The method of claim 1, wherein said administering inhibits base excision repair (BER), nucleotide excision repair (NER) or double stranded DNA break repair in said subject.

13. The method of claim 1, wherein said administering increases γ-H2AX protein activity or expression in said subject.

14. The method of claim 1, wherein said administering lowers Rad51 protein activity or expression in said subject.

15. The method of claim 1, wherein said administering lowers BRCA1 protein activity or expression in said subject.

16. The method of claim 1, wherein said administering lowers PARP1 protein activity or expression in said subject.

17. A pharmaceutical composition comprising a pharmaceutically acceptable excipient and a compound having the structure: 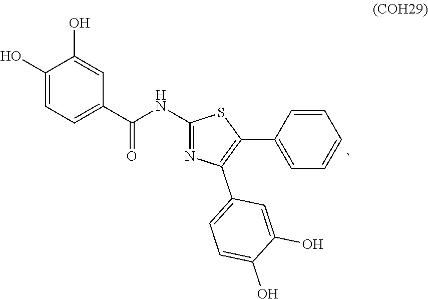 [SMILES: O=C(Nc1nc(-c2ccc(O)c(O)c2)c(-c2ccccc2)s1)c1ccc(O)c(O)c1]
wherein said compound is present in an amount of at least about 50 mg.

18. The pharmaceutical composition of claim 17, wherein said effective amount is from about 50 mg to about 1000 mg.

19. (canceled)

20. The method of claim 1, wherein said subject is a hematological cancer subject.

21. The method of claim 20, wherein said subject is a leukemia cancer subject.


SEQUENCE LISTING (4 sequences)

SEQ ID NO: 1 - RNA, 19 nt, Artificial sequence
  Synthetic polynucleotide
ucacaguguccuuuaugua

SEQ ID NO: 2 - RNA, 19 nt, Artificial sequence
  Synthetic polynucleotide
uacauaaaggacacuguga

SEQ ID NO: 3 - DNA, 24 bp, Artificial sequence
  Synthetic polynucleotide
aggaattgcgggaggaaaatgggt

SEQ ID NO: 4 - DNA, 24 bp, Artificial sequence
  Synthetic polynucleotide
gccccctgaagatctttctgtcct